(19) United States
(12) Patent Application Publication
(10) Pub. No.: US 2023/0314411 A1
(43) Pub. Date: Oct. 5, 2023

(54) METHODS AND KITS OF ASSESSING STATUS, RISK OR PROGNOSIS OF TYPE 1 DIABETES

(71) Applicants: INSERM (INSTITUT NATIONAL DE LA SANTÉ ET DE LA RECHERCHE MÉDICALE), Paris (FR); UNIVERSITÉ PARIS DESCARTES, Paris (FR); CENTRE NATIONAL DE LA RECHERCHE SCIENTIFIQUE (CNRS), Paris (FR)

(72) Inventors: Agnès LEHUEN-MONTEIRO, Paris (FR); Isabelle NEL, Paris (FR); Jennifer DA SILVA, Paris (FR); Lucie BEAUDOIN, Paris (FR)

(21) Appl. No.: 18/331,997

(22) Filed: Jun. 9, 2023

Related U.S. Application Data
(63) Continuation of application No. 16/129,936, filed on Sep. 13, 2018, now abandoned.
Application No. 16/129,936 is a continuation-in-part of application No. PCT/EP2017/056859, filed on Mar. 22, 2017.

(30) Foreign Application Priority Data
Mar. 22, 2016 (EP) 16305319.2

(51) Int. Cl. G01N 33/50; G01N 33/68; A61P 3/02; A61P 3/10

(52) U.S. Cl. CPC G01N 33/505; G01N 33/6893; A61P 3/02; A61P 3/10; G01N 2800/042; G01N 2800/50; G01N 2800/60

(57) ABSTRACT

The present invention relates to methods and kits of assessing status, risk or prognosis of type 1 diabetes. There is still a need for improved methods of prognosis of type 1 diabetes. The inventors have observed different alterations of iNKT and MAIT cells quantity, frequency and markers in T1D patients compared to controls and also in children with recent onset T1D compared to control children or children with established T1D. The present invention relates to a method of assessing status, risk or prognosis of type 1 diabetes in a subject comprising i) quantifying at least one population of innate-like T-cells in a blood sample obtained from the subject, ii) comparing the quantification value determined at step i) with a predetermined reference value and iii) detecting differential in the quantification value determined at step i) and the predetermined reference value is indicative of the status, risk of prognosis of type 1 diabetes.

### FIELD OF THE INVENTION

The present invention relates to methods and kits of assessing status, risk or prognosis of type 1 diabetes.

### BACKGROUND OF THE INVENTION

Type 1 diabetes (TID) is an autoimmune disease that results from the selective destruction of insulin-producing β-cells in pancreatic islets. The diagnosis of TID is commonly preceded by a long prodromal period which includes seroconversion to islet autoantibody positivity and subtle metabolic disturbances. Thus there is still a need for improved methods of assessing status, risk or prognosis of type 1 diabetes. The incidence of TID among children and adolescents has increased markedly in the Western countries during the recent decades and is presently increasing at a faster rate than ever before. This suggests an important role of environment and gene-environment interactions in TID pathogenesis. It is also hypothesized that gut microbiota may affect its incidence via the modulation of the host innate immune system. In particular, the literature describes a decreased diversity in the intestinal microbiota in at risk children with seroconversions who progressed to T1D compared to those who did not progress during the study observation period (Kostic et al, Cell Host & Microbes 2015). Because this observation takes place in the time window between the first seroconversion and much before the T1D diagnosis, it suggests that an event linked to the intestinal microbiota could contribute to the progression of islet autoimmunity towards clinical T1D. Moreover, this decrease of intestinal microbiota diversity seems to be associated with an increased intestinal permeability in at risk children who have at least two autoantibodies (Bosi E et al, Diabetologia 2006 and Graham S et al, Gut 2004). Recently published data also show modification of glycolipids in the stools of at risk children (Kostic et al, Cell Host & Microbes 2015).

### SUMMARY OF THE INVENTION

The present invention relates to methods and kits of assessing status, risk or prognosis of type 1 diabetes. In particular, the present invention is defined by the claims.

### DETAILED DESCRIPTION OF THE INVENTION

The present invention relates to a method of assessing status, risk or prognosis of type 1 diabetes in a subject comprising i) quantifying at least one population of innate-like T-cells in a blood sample obtained from the subject, ii) comparing the quantification value determined at step i) with a predetermined reference value and iii) detecting differential in the quantification value determined at step i) and the predetermined reference value is indicative of the status, risk or prognosis of type 1 diabetes.

In some embodiments, the subject is child. In some embodiments the child is 3 to 5 years old.

In some embodiments, the subject is an adult. In some embodiments, the adult is more than 18 years old.

In some embodiments, the method of the present invention is suitable for identifying whether a subject has or is at risk of having type 1 diabetes (i.e. status or risk) in particular in children. In some embodiments, the method of the present invention is suitable for determining the outcome of type 1 diabetes (i.e. prognosis) in particular in adults.

In some embodiments, the population of innate-like T-cells is a population of MAIT cells. As used herein, the term “MAIT” cells” or “Mucosal-Associated Invariant T cells” refers to a population of T cells that display an invariant TCR alpha chain comprising Vα7.2-Joc33, a CDR3 of constant length, and a limited number of Vβ segments (see, e.g., Lantz and Bendelac. 1994. J. Exp Med. 180:1097-106; Tilloy et al., J. Exp. Med., 1999, 1907-1921; Treiner et al. (2003) Nature 422:164-169, the entire disclosures of each of which are herein incorporated by reference). MAIT cells are restricted by the non-classical MHC class I molecule MRl.

In some embodiments, the population of innate-like T-cells is a population of iNKT cells. As used herein, the term “iNKT cells” or “invariant NKT cells” refers to a major subset of NKT cells, also called type 1 NKT cells expressing an invariant natural T cell receptor (TCR) composed of V[alpha] 14-J[alpha] 18 chains in mice (V[alpha]24-J[alpha] 18 in humans). Upon TCR stimulation with a ligand, such as alpha-galactosylceramide, iNKT cells rapidly produce a wide range of cytokines including IL-4, IFN-[gamma], IL-12, and GM-CSF. It should be further noted that iNKT cells comprise two main subsets (or subpopulations), namely CD4+ and CD4− cells, which in humans have distinct cytokine secretion profiles. This rapid and potent response to a ligand enables iNKT cells to enhance or regulate the activity of various immune cells in innate and acquired immunity.

In some embodiments, the population of innate-like T-cells is characterized by the presence or absence of at least one marker of activation, proliferation, exhaustion or cytotoxicity. In some embodiments, the population of innate-like T-cells is characterized by the presence or absence of the expression of at least one cytokine. In some embodiments, the population of innate-like T-cells of the present invention is characterized by the presence or absence of at least one marker selected from the group consisting of CCR6, CD56, CD25, CD69, CD161, CD27, PD1, TIM3, KLRG1, BTLA, BCL2, Ki67, CD127, GzB, IFNg, IL2, TNFa, IL4, IL10 and IL17.

As used herein the term “CCR6” has its general meaning in the art and refers to chemokine (C-C motif) receptor 6 (Gene ID: 1235). CCR6 is also known as BN-1; DCR2; DRY6; CCR-6; CD196; CKRL3; GPR29; CKR-L3; CMKBR6; GPRCY4; STRL22; CC-CKR-6; and C-C CKR-6.

As used herein the term “CD56” has its general meaning in the art and refers to the neural cell adhesion molecule 1 (Gene ID: 4684). CD56 is also known as MSK39 and NCAM.

As used herein the term “CD161” has its general meaning in the art and refers to the killer cell lectin like receptor B1 (Gene ID: 3820). CD161 is also known as KLRB1, NKR; CD161; CLEC5B; NKR-P1; NKRP1A; NKR-P1A; hNKR-P1A.

As used herein the term “CD27” has its general meaning in the art and refers to the CD27 molecule (Gene ID:939). CD27 is also known as S152, S152. LPFS2, T14, TNFRSF7, and Tp55.

As used herein the term “CD25” has its general meaning in the art and refers to the interleukin 2 receptor subunit alpha (Gene ID: 3559). CD25 is also known as IL2RA, p55; IL2R; IMD41; TCGFR; and IDDM10.

As used herein the term “CD69” has its general meaning in the art and refers to the CD69 molecule (Gene ID: 969). CD69 is also known as AIM, BL-AC/P26, CLEC2C, EA1, GP32/28, and MLR-3.

As used herein the term “PD1” has its general meaning in the art and refers to the programmed cell death 1 (Gene ID: 5133). PD1 is also known as PDCD1, CD279, PD-1, SLEB2, hPD-1, hPD-1, and hSLE1.

As used herein the term “TIM3” has its general meaning in the art and refers to the hepatitis A virus cellular receptor 2 (Gene ID: 84868). TIM3 is also known as HAVCR2, CD366, HAVcr-2, KIM-3, TIMD-3, TIMD3, and Tim-3.

As used herein the term “KLRG1” has its general meaning in the art and refers to the killer cell lectin like receptor G1 (Gene ID: 10219). KLRG1 is also known as 2F1, CLEC15A, MAFA, MAFA-2F1, MAFA-L, and MAFA-LIKE.

As used herein the term “BTLA” has its general meaning in the art and refers to the B and T lymphocyte associated (Gene ID: 151888). BTLA is also known as CD272.

As used herein the term “BCL2” has its general meaning in the art and refers to the B-cell CLL/lymphoma 2 (Gene ID: 596). BCL2 is also known as Bcl-2 and PPP1R50.

As used herein the term “Ki67” has its general meaning in the art and refers to the marker of proliferation Ki-67 (Gene ID: 4288). Ki67 is also known as MKI67, KIA, MIB-, MIB-1, and PPP1R105.

As used herein the term “CD127” has its general meaning in the art and refers to the interleukin 7 receptor (Gene ID: 3575). CD127 is also known as CDW127, IL-7R-alphaA, ILRA, and IL7R.

As used herein the term “GzB” has its general meaning in the art and refers to the granzyme B (Gene ID: 3002). GZB is also known as CCPI, CGL-1, CGL1, CSP-B, CSPB, CTLA1, CTSGL1, HLP, and SECT.

As used herein the term “IFNg” has its general meaning in the art and refers to the interferon, gamma (Gene ID: 3458). IFNg is also known as IFG and IFI.

As used herein the term “IL2” has its general meaning in the art and refers to the interleukin 2 (Gene ID: 3558). IL2 is also known as IL-2, TCGF and lymphokine.

As used herein the term “TNFa” has its general meaning in the art and refers to the tumor necrosis factor (Gene ID: 7124). TNFa is also known as DIF-alpha, TNFA, TNFSF2, TNLG1F, and TNF.

As used herein the term “IL4” has its general meaning in the art and refers to the interleukin 4 (Gene ID: 3565). IL4 is also known as BCGF-1, BCGF1, BSF-1, BSF1, and IL-4.

As used herein the term “IL10” has its general meaning in the art and refers to the interleukin 10 (Gene ID: 3586). IL10 is also known as CSIF, GVHDS, IL-10A, TGIF, and IL10.

As used herein the term “IL17” has its general meaning in the art and refers to the interleukin 17A (Gene ID: 3605). IL17 is also known as CTLA-8, CTLA8, IL-17, IL-17A, and IL17A.

In some embodiments, the method of the present invention comprises quantifying at least one population of MAIT cells expressing 1, 2, 3, 4, 5, or 6 markers selected from the group consisting of CCR6, CD56, CD25, CD69, PD1, TIM3, CD27, KLRG1, BTLA, BCL2, Ki67, CD127, GzB, IFNg, IL2, TNFa, IL4, IL10 and IL17.

In some embodiments, the method of the present invention comprises quantifying at least one population selected from the group consisting of MAIT BTLA+, MAIT CCR6+, MAIT CD25+, MAIT CD56+, MAIT CD69+, MAIT CD127+, MAIT PD1+, MAIT CD27−, MAIT KLRG1+, MAIT TIM3+, iNKT, iNKT BTLA+, iNKT CCR6+, iNKT CD25+, iNKT CD56+, iNKT CD69+, iNKT CD127+, iNKT CD161+, iNKT PD1+, iNKT CD27−, iNKT KLRG1+, and iNKT TIM3+.

In particular, the method of the present invention comprises quantifying at least one population selected from the group consisting of MAIT, MAIT CCR6+, MAIT CD25+, MAIT CD56+, MAIT PD1+, iNKT CCR6+, iNKT CD25+ and iNKT PD1+

More particularly, the method of the present invention comprises quantifying at least one population selected from the group consisting of MAIT, MAIT CCR6+, MAIT CD25+, MAIT CD56+ and MAIT PD1+.

In some embodiments, the method of the present invention comprises quantifying at least one population selected from the group consisting of MAIT BCL2+, MAIT Ki67+, MAIT GzB+, MAIT IFNg+, MAIT IL2+, MAIT IL4+, MAIT IL10+, MAIT IL17+, MAIT TNFa+, iNKT BCL2+, iNKT Ki67+, iNKT GzB+, iNKT IFNg+, iNKT IL2+, iNKT IL4+, iNKT IL10+, iNKT IL17+, and iNKT TNFa+.

In some embodiments, the method of the present invention comprises quantifying at least one population selected from the group consisting of MAIT CCR6+CD56+, MAIT CCR6-CD56−, MAIT CCR6+CD25+, MAIT CCR6+CD25−, MAIT CCR6+PD1−, MAIT CD25+CD56−, MAIT CD25+CCR6−, MAIT CD25+PD1+, MAIT CD25+PD1−, MAIT CD25−PD1−, MAIT CD56+PD1−, MAIT CD56−PD1+, MAIT CD56−PD1−, MAIT CCR6−PD1+, MAIT CD25+CD56−PD1+, MAIT CD25+CD56−PD1−, MAIT CCR6+CD25+PD1+, MAIT CCR6+CD25+PD1−, MAIT CCR6+CD25−PD1−, MAIT CCR6−CD25+PD1−, MAIT CCR6+CD25+CD56−, MAIT CCR6−CD25+CD56−, MAIT CCR6+CD56+PD1−, MAIT CCR6+CD25+CD56−PD1+, MAIT CCR6+CD25+CD56−PD1−, MAIT CCR6−CD25+CD56−PD1−, iNKT CCR6+CD161−, iNKT CD25+PD1+, iNKT CD25−CD161+, iNKT CD69+CD161+, iNKT CD69+CD161−, iNKT CD69−PD1+, iNKT CD161+PD1−, iNKT CCR6−CD25+CD161+, iNKT CD25−CD161+PD1−, iNKT CCR6+CD25−CD161−PD1+, iNKT CCR6−CD25+CD161+PD1+, and iNKT CCR6−CD25+CD161+PD1−.

In particular, the method of the present invention comprises quantifying at least one population selected from the group consisting of MAIT CD25+PD1+, MAIT CD25+PD1−, MAIT CD25+CCR6−, MAIT CCR6+CD56+, MAIT CCR6−CD56−, MAIT CD25+CD56−PD1+, MAIT CCR6−CD25+PD1−, iNKT CCR6−CD25+CD161+, MAIT CCR6+CD25+CD56−, MAIT CCR6+CD25+PD1−, MAIT CCR6+CD25+PD1+, MAIT CCR6−CD25+CD56−, MAIT CD25+CD56−PD1−, MAIT CCR6+CD25+CD56−PD1+, MAIT CCR6+CD25+CD56−PD1−, MAIT CCR6−CD25+CD56−PD1−, iNKT CCR6−CD25+CD161+PD1−, and iNKT CCR6−CD25+CD161+PD1+.

More particularly, the method of the present invention comprises quantifying at least one population selected from the group consisting of MAIT CCR6+CD56+, MAIT CCR6−CD56−, MAIT CD25+PD1+, and MAIT CD25+CD56−PD1+.

In some embodiments, the method of the present invention comprises quantifying at least one population selected from the group consisting of MAIT IFNg+IL4+, MAIT IFNg-IL4−, MAIT IFNg+ GrB−, MAIT IFNg-GrB+, MAIT IFNg+IL17−, MAIT IFNg-IL17+, MAIT IFNg-IL17−, MAIT IL4+ GrB−, MAIT IL4− GrB+, MAIT IL17+ GrB+, MAIT IL17− GrB−, MAIT GrB− IFNg+IL4+, MAIT GrB− IFNg+IL4−, and MAIT GrB+ IFNg− IL4−.

In some embodiments, when the subject is a child, the method of the present invention comprises:
i) quantifying the population of MAIT cells in the blood sample obtained from the subject, ii) comparing the quantification value determined at step i) with a predetermined reference value and iii) concluding that the subject has or is at risk of having type 1 diabetes when the quantification value at step i) is lower than the predetermined reference value, or
i) quantifying the population of MAIT CCR6+ cells in the blood sample obtained from the subject, ii) comparing the quantification value determined at step i) with a predetermined reference value and iii) concluding that the subject has or is at risk of having type 1 diabetes when the quantification value at step i) is lower than the predetermined reference value, or
i) quantifying the population of MAIT CD25+ cells in the blood sample obtained from predetermined reference value and iii) concluding that the subject has or is at risk of having type 1 diabetes when the quantification value at step i) is higher than the predetermined reference value
i) quantifying the population of MAIT CD69+ cells in the blood sample obtained from the subject, ii) comparing the quantification value determined at step i) with a predetermined reference value and iii) concluding that the subject has or is at risk of having type 1 diabetes when the quantification value at step i) is higher than the predetermined reference value, or
i) quantifying the population of MAIT CD56+ cells in the blood sample obtained from the subject, ii) comparing the quantification value determined at step i) with a predetermined reference value and iii) concluding that the subject has or is at risk of having type 1 diabetes when the quantification value at step i) is lower than the predetermined reference value, or
i) quantifying the population of MAIT PD1+ cells in the blood sample obtained from the subject, ii) comparing the quantification value determined at step i) with a predetermined reference value and iii) concluding that the subject has or is at risk of having type 1 diabetes when the quantification value at step i) is higher than the predetermined reference value, or
i) quantifying the population of MAIT TIM3+ cells in the blood sample obtained from the subject, ii) comparing the quantification value determined at step i) with a predetermined reference value and iii) concluding that the subject has or is at risk of having type 1 diabetes when the quantification value at step i) is higher than the predetermined reference value, or
i) quantifying the population of MAIT KLRG1+ cells in the blood sample obtained from the subject, ii) comparing the quantification value determined at step i) with a predetermined reference value and iii) concluding that the subject has or is at risk of having type 1 diabetes when the quantification value at step i) is higher than the predetermined reference value, or
i) quantifying the population of MAIT BTLA+ cells in the blood sample obtained from the subject, ii) comparing the quantification value determined at step i) with a predetermined reference value and iii) concluding that the subject has or is at risk of having type 1 diabetes when the quantification value at step i) is higher than the predetermined reference value, or
i) quantifying the population of MAIT BCL2+ cells in the blood sample obtained from the subject, ii) comparing the quantification value determined at step i) with a predetermined reference value and iii) concluding that the subject has or is at risk of having type 1 diabetes when the quantification value at step i) is lower than the predetermined reference value, or
i) quantifying the population of MAIT Ki67+ cells in the blood sample obtained from the subject, ii) comparing the quantification value determined at step i) with a predetermined reference value and iii) concluding that the subject has or is at risk of having type 1 diabetes when the quantification value at step i) is higher than the predetermined reference value, or
i) quantifying the population of MAIT GzB+ cells in the blood sample obtained from the subject, ii) comparing the quantification value determined at step i) with a predetermined reference value and iii) concluding that the subject has or is at risk of having type 1 diabetes when the quantification value at step i) is higher than the predetermined reference value, or
i) quantifying the population of MAIT IFNg+ cells in the blood sample obtained from the subject, ii) comparing the quantification value determined at step i) with a predetermined reference value and iii) concluding that the subject has or is at risk of having type 1 diabetes when the quantification value at step i) is lower than the predetermined reference value, or
i) quantifying the population of MAIT TNFa+ cells in the blood sample obtained from the subject, ii) comparing the quantification value determined at step i) with a predetermined reference value and iii) concluding that the subject has or is at risk of having type 1 diabetes when the quantification value at step i) is higher than the predetermined reference value, or
i) quantifying the population of MAIT IL4+ cells in the blood sample obtained from predetermined reference value and iii) concluding that the subject has or is at risk of having type 1 diabetes when the quantification value at step i) is higher than the predetermined reference value, or
i) quantifying the population of MAIT IL10+ cells in the blood sample obtained from the subject, ii) comparing the quantification value determined at step i) with a predetermined reference value and iii) concluding that the subject has or is at risk of having type 1 diabetes when the quantification value at step i) is higher than the predetermined reference value, or
i) quantifying the population of MAIT IL17+ cells in the blood sample obtained from the subject, ii) comparing the quantification value determined at step i) with a predetermined reference value and iii) concluding that the subject has or is at risk of having type 1 diabetes when the quantification value at step i) is higher than the predetermined reference value.

In some embodiments, when the subject is an adult, the method of the present invention comprises:
i) quantifying the population of MAIT cells in the blood sample obtained from the subject, ii) comparing the quantification value determined at step i) with a predetermined reference value and iii) concluding that the subject has or is at risk of having type 1 diabetes when the quantification value at step i) is lower than the predetermined reference value, or
i) quantifying the population of MAIT CD25+ cells in the blood sample obtained from the subject, ii) comparing the quantification value determined at step i) with a predetermined reference value and iii) concluding that the subject has or is at risk of having type 1 diabetes when the quantification value at step i) is higher than the predetermined reference value
i) quantifying the population of MAIT CD69+ cells in the blood sample obtained from the subject, ii) comparing the quantification value determined at step i) with a predetermined reference value and iii) concluding that the subject has or is at risk of having type 1 diabetes when the quantification value at step i) is higher than the predetermined reference value, or
i) quantifying the population of MAIT CD56+ cells in the blood sample obtained from the subject, ii) comparing the quantification value determined at step i) with a predetermined reference value and iii) concluding that the subject has or is at risk of having type 1 diabetes when the quantification value at step i) is higher than the predetermined reference value, or
i) quantifying the population of MAIT Ki67+ cells in the blood sample obtained from the subject, ii) comparing the quantification value determined at step i) with a predetermined reference value and iii) concluding that the subject has or is at risk of having type 1 diabetes when the quantification value at step i) is higher than the predetermined reference value, or
i) quantifying the population of MAIT IFNg+ cells in the blood sample obtained from the subject, ii) comparing the quantification value determined at step i) with a predetermined reference value and iii) concluding that the subject has or is at risk of having type 1 diabetes when the quantification value at step i) is lower than the predetermined reference value, or
i) quantifying the population of MAIT TNFa+ cells in the blood sample obtained from the subject, ii) comparing the quantification value determined at step i) with a predetermined reference value and iii) concluding that the subject has or is at risk of having type 1 diabetes when the quantification value at step i) is lower than the predetermined reference value.

In some embodiments, when the subject is a child or an adult, the method of the present invention comprises:
i) quantifying the population of iNKT cells in the blood sample obtained from the subject, ii) comparing the quantification value determined at step i) with a predetermined reference value and iii) concluding that the subject has or is at risk of having type 1 diabetes when the quantification value at step i) is lower than the predetermined reference value, or
i) quantifying the population of iNKT CD25+ cells in the blood sample obtained from predetermined reference value and iii) concluding that the subject has or is at risk of having type 1 diabetes when the quantification value at step i) is higher than the predetermined reference value, or
i) quantifying the population of iNKT CD161+ cells in the blood sample obtained from the subjected, ii) comparing the quantification value determined at step i) with a predetermined reference value and iii) concluding that the subject has or is at risk of having type 1 diabetes when the quantification value at step i) is lower than the predetermined reference value, or
i) quantifying the population of iNKT CD69+ cells in the blood sample obtained from the subject, ii) comparing the quantification value determined at step i) with a predetermined reference value and iii) concluding that the subject has or is at risk of having type 1 diabetes when the quantification value at step i) is lower than the predetermined reference value, or
i) quantifying the population of iNKT PD1+ cells in the blood sample obtained from the subject, ii) comparing the quantification value determined at step i) with a predetermined reference value and iii) concluding that the subject has or is at risk of having type 1 diabetes when the quantification value at step i) is higher than the predetermined reference value, or
i) quantifying the population of iNKT KLRG1+ cells in the blood sample obtained from the subject, ii) comparing the quantification value determined at step i) with a predetermined reference value and iii) concluding that the subject has or is at risk of having type 1 diabetes when the quantification value at step i) is higher than the predetermined reference value, or
i) quantifying the population of iNKT BCL2+ cells in the blood sample obtained from the subject, ii) comparing the quantification value determined at step i) with a predetermined reference value and iii) concluding that the subject has or is at risk of having type 1 diabetes when the quantification value at step i) is lower than the predetermined reference value,
i) quantifying the population of iNKT CD127+ cells in the blood sample obtained from the subject, ii) comparing the quantification value determined at step i) with a predetermined reference value and iii) concluding that the subject has or is at risk of having type 1 diabetes when the quantification value at step i) is lower than the predetermined reference value,
i) quantifying the population of iNKT IFNg+ cells in the blood sample obtained from the subject, ii) comparing the quantification value determined at step i) with a predetermined reference value and iii) concluding that the subject has or is at risk of having type 1 diabetes when the quantification value at step i) is lower than the predetermined reference value,
i) quantifying the population of iNKT IL2+ cells in the blood sample obtained from the subject, ii) comparing the quantification value determined at step i) with a predetermined reference value and iii) concluding that the subject has or is at risk of having type 1 diabetes when the quantification value at step i) is lower than the predetermined reference value,
i) quantifying the population of iNKT TNFa+ cells in the blood sample obtained from the subject, ii) comparing the quantification value determined at step i) with a predetermined reference value and iii) concluding that the subject has or is at risk of having type 1 diabetes when the quantification value at step i) is lower than the predetermined reference value.

In some embodiments, at least 2, 3, 4, 5, 6, 7, 8, 9, or 10 population of cells are quantified. In some embodiments, the method of the present invention comprises quantifying the population of MAIT CCR6+, MAIT CD56+, MAIT CCR6+CD56+, MAIT CCR6− CD56−, MAIT CD25+PD1+ and MAIT CD25+CD56− PD1+.

In some embodiments, the blood sample is a PBMC sample. As used herein, the term “PBMC” or “peripheral blood mononuclear cells” or “unfractionated PBMC” refers to whole PBMC, i.e. to a population of white blood cells having a round nucleus, which has not been enriched for a given sub-population. Typically, these cells can be extracted from whole blood using Ficoll, a hydrophilic polysaccharide that separates layers of blood, with the PBMC forming a cell ring under a layer of plasma. Additionally, PBMC can be extracted from whole blood using a hypotonic lysis which will preferentially lyse red blood cells. Such procedures are known to the expert in the art.

In some embodiments, the quantification is absolute or relative. In some embodiments, when the quantification is relative, it consists in determining the frequency of the population in the general population of T cells (i.e. characterized by the expression of CD3) or the frequency of the population in the population of MAIT or iNKT cells.

The quantification of the population of innate-like T-cells is determined by any method well known in the art and typically involves flow cytometry methods. As used herein, the term “flow cytometric method” refers to a technique for counting cells of interest, by suspending them in a stream of fluid and passing them through an electronic detection apparatus. Flow cytometric methods allow simultaneous multiparametric analysis of the physical and/or chemical parameters of up to thousands of events per second, such as fluorescent parameters. Modern flow cytometric instruments usually have multiple lasers and fluorescence detectors. A common variation of flow cytometric techniques is to physically sort particles based on their properties, so as to purify or detect populations of interest, using “fluorescence-activated cell sorting”. As used herein, “fluorescence-activated cell sorting” (FACS) refers to a flow cytometric method for sorting a heterogeneous mixture of cells from a biological sample into two or more containers, one cell at a time, based upon the specific light scattering and fluorescent characteristics of each cell and provides fast, objective and quantitative recording of fluorescent signals from individual cells as well as physical separation of cells of particular interest. Accordingly, FACS can be used with the methods described herein to isolate and detect the population of cells of the present invention. For example, fluorescence activated cell sorting (FACS) may be therefore used. It involves using a flow cytometer capable of simultaneous excitation and detection of multiple fluorophores, such as a BD Biosciences FACSCanto™ flow cytometer, used substantially according to the manufacturer's instructions. The cytometric systems may include a cytometric sample fluidic subsystem, as described below. In addition, the cytometric systems include a cytometer fluidically coupled to the cytometric sample fluidic subsystem. Systems of the present disclosure may include a number of additional components, such as data output devices, e.g., monitors, printers, and/or speakers, softwares (e.g. (Flowjo, Kaluza . . .), data input devices, e.g., interface ports, a mouse, a keyboard, etc., fluid handling components, power sources, etc. More particularly, the blood sample is contacted with a panel of antibodies specific for the specific market of the population of cells of the interest. As used herein, the term “antibody” refers to an intact immunoglobulin or to a monoclonal or polyclonal antigen-binding fragment with the Fc (crystallizable fragment) region or FcRn binding fragment of the Fc region. Antigen-binding fragments may be produced by recombinant DNA techniques or by enzymatic or chemical cleavage of intact antibodies. “Antigen-binding fragments” include, inter alia, Fab, Fab′, F(ab′)2, Fv, dAb, and complementarity determining region (CDR) fragments, single-chain antibodies (scFv), single domain antibodies, chimeric antibodies, diabodies and polypeptides that contain at least a portion of an immunoglobulin that is sufficient to confer specific antigen binding to the polypeptide. The terms Fab, Fc, pFc′, F(ab′) 2 and Fv are employed with standard immunological meanings (Roitt, I. (1991) Essential Immunology, 7th Ed., (Blackwell Scientific Publications, Oxford)]. Such antibodies or antigen-binding fragments are available commercially from vendors such as R&D Systems, BD Biosciences, e Biosciences, Biolegend, Proimmune and Miltenyi, or can be raised against these cell-surface markers by methods known to those skilled in the art. In some embodiments, an agent that specifically bind to a cell-surface marker, such as an antibody or antigen-binding fragment, is labelled with a tag to facilitate the isolation and detection of population of cells of the interest. As used herein, the terms “label” or “tag” refer to a composition capable of producing a detectable signal indicative of the presence of a target, such as, the presence of a specific cell-surface marker in a biological sample. Suitable labels include fluorescent molecules, radioisotopes, nucleotide chromophores, enzymes, substrates, chemiluminescent moieties, magnetic particles, bioluminescent moieties, and the like. As such, a label is any composition detectable by spectroscopic, photochemical, biochemical, immunochemical, electrical, optical or chemical means needed for the methods to isolate and detect immune cells or iNKT cell populations and MAIT cell populations. Non-limiting examples of fluorescent labels or tags for labeling the agents such as antibodies for use in the methods of invention include Hydroxycoumarin, Succinimidyl ester, Aminocoumarin, Succinimidyl ester, Methoxycoumarin, Succinimidyl ester, Cascade Blue, Hydrazide, Pacific Blue, Maleimide, Pacific Orange, Lucifer yellow, NBD, NBD-X, R-Phycoerythrin (PE), a PE-Cy5 conjugate (Cychrome, R670, Tri-Color, Quantum Red), a PE-Cy7 conjugate, Red 613, PE-Texas Red, PerCP, PerCPeFluor 710, PE-CF594, Peridinin chlorphyll protein, TruRed (PerCP-Cy5.5 conjugate), FluorX, Fluoresceinisothyocyanate (FITC), BODIPY-FL, TRITC, X-Rhodamine (XRITC), Lissamine Rhodamine B, Texas Red, Allophycocyanin (APC), an APC-Cy7 conjugate, Alexa Fluor 350, Alexa Fluor 405, Alexa Fluor 430, Alexa Fluor 488, Alexa Fluor 500, Alexa Fluor 514, Alexa Fluor 532, Alexa Fluor 546, Alexa Fluor 555, Alexa Fluor 568, Alexa Fluor 594, Alexa Fluor 610, Alexa Fluor 633, Alexa Fluor 647, Alexa Fluor 660, Alexa Fluor 680, Alexa Fluor 700, Alexa Fluor 750, Alexa Fluor 790, Cy2, Cy3, Cy3B, Cy3.5, Cy5, Cy5.5, Cy7, BV 785, BV711, BV421, BV605, BV510 or BV650. The aforementioned assays may involve the binding of the antibodies to a solid support. The solid surface could be a microtitration plate coated with the antibodies. Alternatively, the solid surfaces may be beads, such as activated beads, magnetically responsive beads. Beads may be made of different materials, including but not limited to glass, plastic, polystyrene, and acrylic. In addition, the beads are preferably fluorescently labelled. In a preferred embodiment, fluorescent beads are those contained in TruCount™ tubes, available from Becton Dickinson Biosciences, (San Jose, California). In some embodiments, PBMC were stained for detection of cytokines production after stimulation in RPMI medium supplemented with 10% fetal bovine serum with PMA and ionomycin at ng/mL and 1 μg/mL, respectively, in the presence of brefeldin A at 10 μg/mL for 6 hours at 37° C. As being intra cellularly located, cytokine expression may be assessed by intracellular flow cytometry. Intracellular flow cytometry typically involves the permeabilization and fixation of the cells. Any convenient means of permeabilizing and fixing the cells may be used in practicing the methods. For example permeabilizing agent typically include saponin, methanol, Tureen® 20, Triton X-100™

Typically, the predetermined reference value is a threshold value or a cut-off value. Typically, a “threshold value” or “cut-off value” can be determined experimentally, empirically, or theoretically. A threshold value can also be arbitrarily selected based upon the existing experimental and/or clinical conditions, as would be recognized by a person of ordinary skilled in the art. For example, retrospective measurement in properly banked historical subject samples may be used in establishing the predetermined reference value. The threshold value has to be determined in order to obtain the optimal sensitivity and specificity according to the function of the test and the benefit/risk balance (clinical consequences of false positive and false negative). Typically, the optimal sensitivity and specificity (and so the threshold value) can be determined using a Receiver Operating Characteristic (ROC) curve based on experimental data. For example, after determining the quantification of the selected population in a group of reference, one can use algorithmic analysis for the statistic treatment of the expression levels determined in samples to be tested, and thus obtain a classification standard having significance for sample classification. The full name of ROC curve is receiver operator characteristic curve, which is also known as receiver operation characteristic curve. It is mainly used for clinical biochemical diagnostic tests. ROC curve is a comprehensive indicator that reflects the continuous variables of true positive rate (sensitivity) and false positive rate (1-specificity). It reveals the relationship between sensitivity and specificity with the image composition method. A series of different cut-off values (thresholds or critical values, boundary values between normal and abnormal results of diagnostic test) are set as continuous variables to calculate a series of sensitivity and specificity values. Then sensitivity is used as the vertical coordinate and specificity is used as the horizontal coordinate to draw a curve. The higher the area under the curve (AUC), the higher the accuracy of diagnosis. On the ROC curve, the point closest to the far upper left of the coordinate diagram is a critical point having both high sensitivity and high specificity values. The AUC value of the ROC curve is between 1.0 and 0.5. When AUC>0.5, the diagnostic result gets better and better as AUC approaches 1. When AUC is between 0.5 and 0.7, the accuracy is low. When AUC is between 0.7 and 0.9, the accuracy is moderate. When AUC is higher than 0.9, the accuracy is high. This algorithmic method is preferably done with a computer. Existing software or systems in the art may be used for the drawing of the ROC curve, such as: MedCalc 9.2.0.1 medical statistical software, SPSS 9.0, ROCPOWER.SAS, DESIGNROC.FOR, MULTIREADER POWER.SAS, CREATE-ROC.SAS, GB STAT VI0.0 (Dynamic Microsystems, Inc. Silver Spring, Md., USA), etc.

In some embodiments, when more than 1 population of cells are quantified, method of the present invention comprises calculating a composite score that integrates the weight for each population in the contribution in the risk and comparing the patient's composite score to one or more predetermined reference values for determining whether the patient has or is at risk of having T1D. In some embodiments, the method of the present invention comprises calculating a composite score that integrates the weight for the following populations: MAIT CCR6+, MAIT CD56+, MAIT CCR6+CD56+, MAIT CCR6− CD56−, MAIT CD25+PD1+ and MAIT CD25+CD56− PD1+.

A further object of the invention relates to a kit comprising means for performing the method of the present invention. Typically, the kit comprises means for quantifying the population of cells. In some embodiments, said means are antibodies as described above. In some embodiments, these antibodies are labelled as described above. Typically, the kits described above will also comprise one or more other containers, containing for example, wash reagents, and/or other reagents capable of quantitatively detecting the presence of bound antibodies. The kit also contains agents suitable for performing intracellular flow cytometry such as agents for permeabilization and fixation of cells. Typically compartmentalised kit includes any kit in which reagents are contained in separate containers, and may include small glass containers, plastic containers or strips of plastic or paper. Such containers may allow the efficient transfer of reagents from one compartment to another compartment whilst avoiding cross-contamination of the samples and reagents, and the addition of agents or solutions of each container from one compartment to another in a quantitative fashion. Such kits may also include a container which will accept the blood sample, a container which contains the antibody(s) used in the assay, containers which contain wash reagents (such as phosphate buffered saline, Tris-buffers, and like), and containers which contain the detection reagent.

In addition, the present invention relates to methods of treating a subject in order to prevent the onset of full blown T1D in subjects who are deemed to be at risk of developing the disease. That is, T1D is not yet diagnosed in the subject and major symptoms of the disease are not yet present, but a pre-diabetic condition is indicated so that, in the absence of medical or other intervention, T1D is likely to develop. Medical interventions (therapies) at an early stage include but are not limited to: the administration of one or more agents that preserve, stop the destruction of or regenerate production of β-cells by pancreatic islets (e.g. antibodies which target autoantibodies); or agents or procedures that increase or maintain the production of 0-cells (e.g. pancreatic islet transplant); administration of one or more agents that control blood glucose levels and/or increase insulin production (e.g. sulfonylureas, meglitinides, fructose/honey, dipeptidyl peptidase-4 (DPP-4) inhibitors, pioglitazone); administration of prebiotics and/or probiotics and/or synbiotics to alter gut microbe populiations, usually to increase diversity (e.g. administration of live microorganisms, usually bacteria and/or yeasts, that come in a variety of formulations; and/or gut microbiome transplants such as fecal transplants/transfers); various vaccine strategies to induce tolerance e.g. islet transplantation tolerance induction, administration of: β-cell auto-Ags or their mimotopes such as insulin (INS) and its precursor preproinsulin (PPI), 65 kD glutamic acid decarboxylase (GAD65/GAD2), islet Ag (IA)-2 (PTPRN), zinc transporter 8 (ZnT8/SLC30A8), islet-specific glucose-6-phosphatase catalytic subunit-related protein (IGRP), chromogranin A (CHGA), islet amyloid polypeptide (IAPP), mutated forms of Hsp60, β-cell mimotope peptides, recombinant DNA therapy to manipulate β-cell autoimmunity, autologous chimeric antigen receptor (CAR) T cells may be generated for administration by expressing a β-cell antigen-reactive immunoglobulin scFv providing tissue-targeted activation and eliciting bystander suppression, etc.

In addition, weight management and/or food control intake measures including but not limited to: a prescribed diet e.g. a diet low in carbohydrates, low in foods with a high glycemic index, low in sugars, etc. and a planned exercise regimen, either or both of which may be supervised by a medical professional; administration of weight loss medications (e.g. orlistat (Xenical, Alli), lorcaserin (Belviq), phentermine-topiramate (Qsymia), naltrexone-bupropion (Contrave), or liraglutide (Saxenda)) or agents that suppress appetite (e.g. phentermine, benzphetamine, diethylpropion, phendimetrazine); and the like, may be employed alone or in conjunction with the administration of suitable agents.

Such treatments or therapies may result in a complete or partial reversal of the initial tendancy toward development of T1D so that the subject does not develop T1D, or develops T1D more slowly than would otherwise occur, or develops fewer or less serious or severe symptoms of T1D.

In addition, the present invention relates to a method for treating T1D in a subject in need thereof (for example, a subject who is diagnosed with T1D using the methods described herein). The methods or therapies comprise administering to the subject a suitable, therapeutically effective amount of one or more agents that lower blood sugar and/or a gene therapy treatment that lowers blood sugar. The diagnosis may also be confirmed by testing and monitoring the blood sugar level of the subject. A random blood sugar level of 200 mg/dL (11.1 mmol/L) or higher suggests diabetes, especially when coupled with any of the signs and symptoms of diabetes, such as frequent urination and extreme thirst. A fasting blood sugar level less than 100 mg/dL (5.6 mmol/L) is normal. A fasting blood sugar level from 100 to 125 mg/dL (5.6 to 6.9 mmol/L) is considered prediabetes. A fasting blood sugar level of 126 mg/dL (7 mmol/L) or higher on two separate tests, confirms diabetes. In addition, A1C levels (glycated hemoglobin levels) may be determined and monitored before and during treatment. When the A1C test is used to diagnose diabetes, an A1C level of 6.5 percent or higher on two separate occasions indicates the subject has diabetes. A result between 5.7 and 6.4 percent is considered prediabetes, which indicates a high risk of developing diabetes. A result below 5.7 is considered normal. For most people who have previously diagnosed diabetes, an A1C. level of 7 percent or less is a common treatment target. Higher targets of up to 8 percent may be appropriate for some individuals. As used herein, the term “subject” denotes a mammal. Typically, a subject according to the invention refers to any subject (preferably human) diagnosed with or afflicted with T1D.

As used herein, the terms “treatment” and “treat” refer to both prophylactic or preventive treatment as well as curative or disease modifying treatment, including treatment of patient at risk of contracting the disease or suspected to have contracted the disease as well as patients who are ill or have been diagnosed as suffering from the disease or medical condition, and includes suppression of clinical relapse. The treatment may be administered to a patient having a medical disorder or who ultimately may acquire the disorder, in order to prevent, cure, delay the onset of, reduce the severity of, or ameliorate one or more symptoms of the disorder or recurring disorder, or in order to prolong the survival of a patient beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular intervals, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

For T1D, the treatment methods typically involve administering a therapeutically effective dose of a treatment, e.g. a drug, that treats the T1D. In some aspects, the drugs is insulin. Many types of insulin are known and include: short-acting (regular) insulin, rapid-acting insulin, intermediate-acting (NPH) insulin, long-acting insulin, etc. In addition, the so-called “artificial pancreas” (closed-loop insulin delivery) for people with type 1 diabetes who are age 14 and older. Pancreas and islet cell transplantation may also be effective. The amount administered is determined by a skilled medical practitioner and is typically sufficient to lower the blood sugar level, preferably to normal or near normal. Insulin is frequently administered together with various other agents such as high blood pressure medications (angiotensin-converting enzyme (ACE) inhibitors or angiotensin II receptor blockers (ARBs)); aspirin; cholesterol-lowering drugs; etc. In addition, one or more of the treatments recommended for those at risk of developing T1D may also be administered, e.g. autologous chimeric antigen receptor (CAR) T cell therapy, changes in diet and exercise, etc.

The invention will be further illustrated by the following figures and examples. However, these examples and figures should not be interpreted in any way as limiting the scope of the present invention.

### FIGURES

FIG. 1: frequencies of MAIT cells in the peripheral blood of control children, T1D recent onset children, control adults and T1D adult patients. Patients correspond to the Table 1.***p<0.001 Mann-Whitney test.

FIG. 2: frequencies of MAIT CCR6+, MAIT CD25+ and MAIT C56+, cells in the peripheral blood of control children, T1D recent onset children, control adults and T1D adult patients. Patients correspond to the Table 1. *p<0.05, **p<0.01, ***p<0.001 Mann-Whitney test.

FIG. 3: frequencies of iNKT CCR6+ and iNKT CD25+ cells in the peripheral blood of control children, T1D recent onset children, control adults and T1D adult patients. Patients correspond to the Table 1. *p<0.05, **p<0.01.

FIG. 4: frequencies of MAIT PD1+ and iNKT PD1+ cells in the peripheral blood of control children and T1D recent onset children. Patients correspond to the Table 1. *p<0.05.

FIG. 5: frequencies of MAIT BCL2+ and MAIT KLRG1+ cells in the peripheral blood of control children and T1D recent onset children. Patients correspond to the Table 1. * p<0.05, **p<0.01.

FIG. 6: frequencies of MAIT IFNg+ and iNKT IFNg+ cells in the peripheral blood of control children, T1D recent onset children. Patients correspond to the Table 1. *p<0.05, **p<0.01.

FIG. 7: frequencies of MAIT TNFa+, MAIT IL4+, MAIT GZB+, and MAIT IL17+ cells in the peripheral blood of control children and T1D recent onset children. Patients correspond to the Table 1. *p<0.05, **p<0.01, ***p<0.001 Mann-Whitney test.

FIG. 8: principal component analysis of cytometric parameters related to MAIT and iNKT cells markers from peripheral blood of control children, T1D recent onset children and T1D children.

FIG. 9: ROC curve for the logistic regression model based on variables MAIT CCR6, MAIT CD56, MAIT CCR6+CD56+, MAIT CCR6− CD56−, MAIT CD25+PD1+ and MAIT CD25+CD56− PD1

FIG. 10A-B: Frequency and phenotype alterations of blood MAIT cells from T1D children. MAIT cells were analyzed in blood from children with recent onset T1D (n=41), children with established T1D (n=23), as compared with control children (n=22). (A) Representative staining of MAIT cells and MAIT cell frequency among T lymphocytes. (B) Representative staining and frequency of MAIT cells expressing different cell surface molecules (CCR6, CD56, CD69, CD25, PD1) and intracellular Bcl-2. P values were determined by Kruskal-Wallis test followed by the Wilcoxon rank sum test adjusted with the Holm method.

FIG. 11A-B: Functional alterations of blood MAIT cells from T1D children. MAIT cells were analyzed in blood from children with recent onset T1D (n=25) and children with established T1D (n=18), as compared with control children (n=18). (A) Representative intracellular staining of MAIT cells for cytokines and GzB after PMA/ionomycin stimulation and graphs showing the frequency of MAIT cells producing cytokines and GzB. P values were determined by Kruskal-Wallis test followed by the Wilcoxon rank sum test adjusted with the Holm method. (B) Frequencies of CD69⁺ and CD25⁺CD69⁺ MAIT cells from children controls (n=6) and children with recent onset T1D (n=7) after ON stimulation with 5-OP-RU at various concentrations (0 to 5 nmol/L). Blocking MR1 mAb was added when indicated. P values were determined by Mann-Whitney test.

FIG. 12A-E: Relationship between MAIT cell characteristics and disease status of patients. (A) Correlations between the age of children with recent onset T1D (n=25) at diagnosis, the frequency of MAIT cells expressing GzB after PMA/Ionomycin stimulation and the HbA1c levels. Correlations between the age and the frequency of MAIT expressing GzB are also shown for the children with established T1D and control children. P value was determined by Spearman test. (B) MAIT cell staining of surface and intracellular molecules performed on PBMC from fifteen children with recent onset T1D at the time of diagnosis (on the left) and around one year after diabetes onset (on the right). P values were determined by Signed-rank Wilcoxon Test. (C) Factorial discriminant analysis based on the expression of surface and intracellular molecules by MAIT cells from the children with recent onset T1D (n=20), the children with established T1D (n=15) and the children controls (n=18). (D) ROC Curve of the predictive model defining the T1D phenotype. (E) FACS Analysis of MAIT cell frequency among T lymphocytes and frequency of MAIT cells expressing different cell surface molecules from frozen PBMC of adult controls (n=11) and adult at risk T1D donors with at least two autoantibodies (n=11). P values were determined by Mann-Whitney test for b and e.

### EXAMPLE 1

We have observed different alterations of iNKT and MAIT cells quantity, frequency and markers in T1D patients compared to controls and it has also been shown that children with recent onset T1D exhibit different alterations of iNKT and MAIT cells compared to control children or children with established T1D. The alterations should suitable of assessing status, risk or prognosis of type 1 diabetes.

Early alterations of these cell populations would suggest their implication in defective immunoregulation occurring at the pre-diabetic stage. As suggested by our data in mice models (NOD mice), both cell populations could represent new opportunities to avoid deleterious immune response against beta cells and develop strategies to prevent diabetes development. Table 1 describes our current cohort. For the analyses presented below we have reanalyzed all data at the same time to ensure that all samples were studied using the same gating strategy, for increased consistency and precision.

| | | | | Adults with |
|---|---|---|---|---|
| | Control | Children with recent | Control | long-standing |
| | Children | onset T1D | Adults | T1D |
| n | 22 | 41 | 29 | 41 |
| Age (Yr) | 9.05 (4.3-18.4) | 8.3 (1.6-16) | 34.9 (23.3-67.3) | 38.9 (17.6-71.1) |
| HbA1c (%) | | 12.1 (8.7-16.5) | | 8.07 (5.5-13.5) |
| Duration of disease | | 4 days (1-10) | | 16.2 years (2-53) |
| BMI/Z-score | −0.3 (−2.2+2.4) | −0.6 (−2.7+3.7) | 21.8 (18.0-27.7) | 23.3 (17.5-37.2) |
| Sex M/F | 13M/9F | 21M/20F | 12M/17F | 15M/26F |

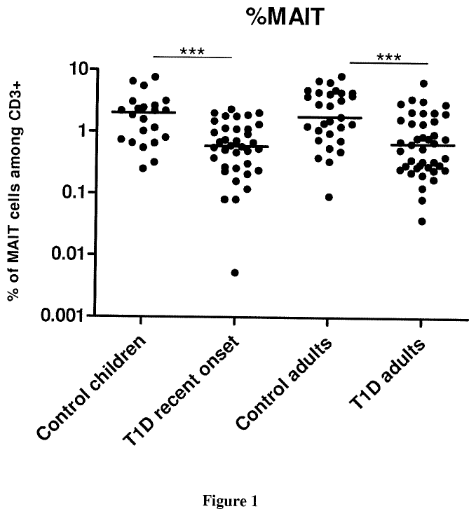

Our results reveal decreased frequencies of iNKT and MAIT cells in children with recent onset T1D (FIG. 1). A reduction of MAIT cells was also seen in adult patients with long-standing T1D compared to adult controls (FIG. 1).

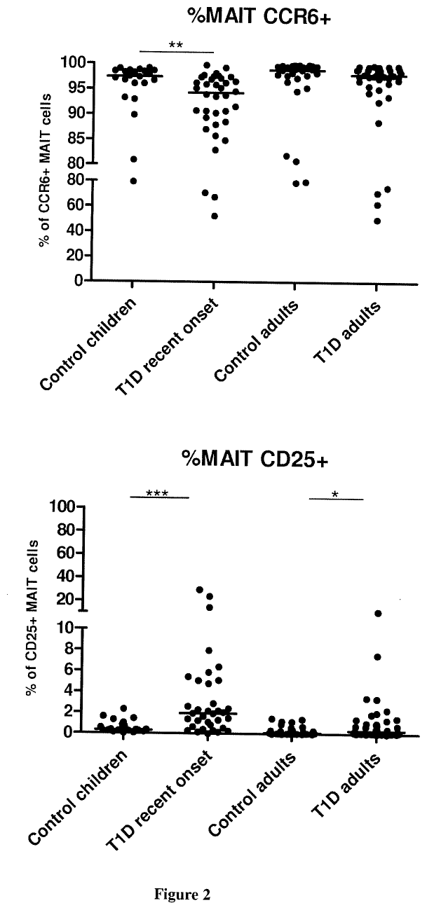

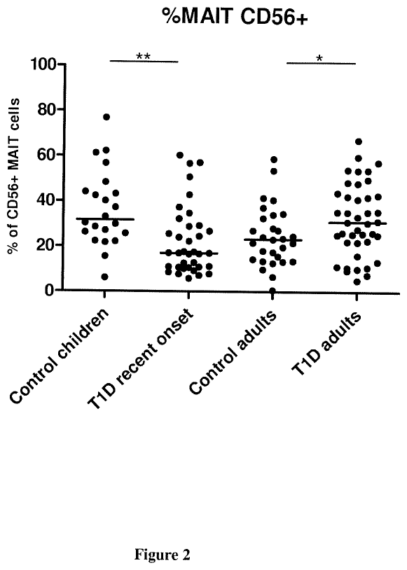

Importantly, a more in depth analysis of the iNKT or MAIT biological markers showed they can be used for the discrimination between controls and patients with recent onset and patient with long-standing T1D. For example, as shown in FIG. 2, the quantification of MAIT CCR6+, MAIT CD25+ or MAIT CD56+ cells can be used for such a discrimination. MAIT cells were more often CD25-positive in patient with T1D, when compared to controls. MAIT cells were more often CCR6-negative in patient with T1D recent onset, compared to controls or adults with long-standing T1D. At last MAIT cells were more often CD56-negative in patient with T1D recent onset and can be used to discriminate it from both controls and adults with long-standing T1D (FIG. 2).

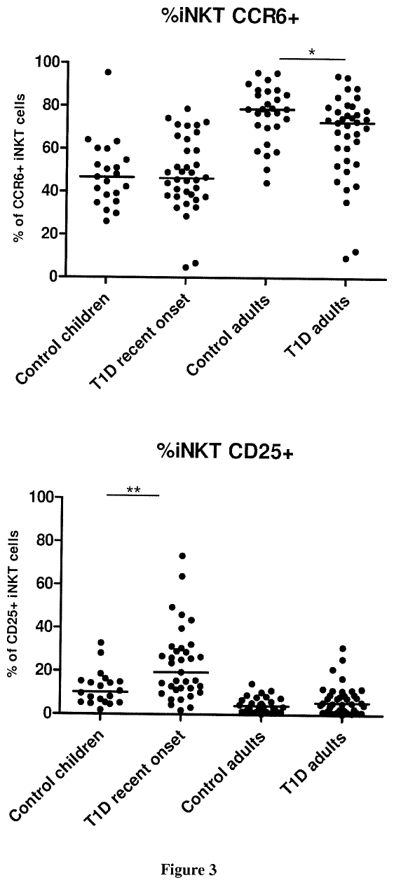

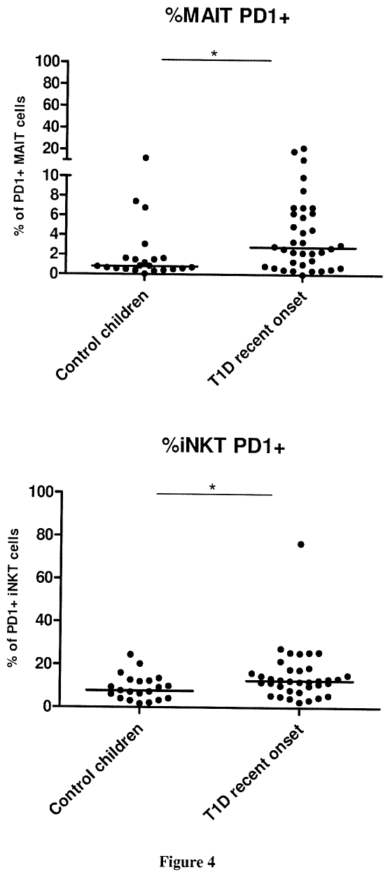

In the same way, iNKT cells were more often CD25-positive in children with recent onset T1D, compared to control children and those with established T1D (FIG. 3) whereas iNKT cells were more often CCR6-negative in adults patients with long-standing T1D compared to controls or recent onset (FIG. 3). A negative correlation between % CD25+ iNKT cells and iNKT cell frequency is also observed in the adult long-standing T1D group (r=−0.50, p=0.002**) and in the children recent onset T1D group (r=−0.35, p=0.04*).

For all of these comparisons, the groups were well matched for age. In short, the most striking finding is a decreased iNKT and MAIT cell frequency in the peripheral blood of recent onset T1D children in association with increased proportions of iNKT and MAIT cells expressing the activation marker CD25. Results have been illustrated in FIGS. 1 to 3 and summarized in Table 2. Of note, there is a positive correlation between the % of CD25+ MAIT cells and the % of CD25+ iNKT cells among the two groups of T1D patients.

| | | | Children | | |
|---|---|---|---|---|---|
| | | Con- | with T1D | Con- | Children |
| | | trols | Recent | trols | with T1D |
| Test Mann-Whitney | | children | Onset | adults | Follow up |
| FRE- | % MAIT | +++ | −−− | +++ | −−− |
| QUENCY | % iNKT | +++ | + | ++ | + |
| MIGRA- | MAIT | +++ | + | +++ | ++ |
| TION | CCR6+ | | | | |
| | MAIT | ++ | −− | + | ++ |
| | CD56+ | | | | |
| | iNKT | + | + | +++ | ++ |
| | CCR6+ | | | | |
| | iNKT CD56+ | + | + | + | +/− |
| ACTI- | MAIT | −−− | +++ | −−− | + |
| VATION | CD25+ | | | | |
| | iNKT CD25+ | + | +++ | − | +/− |

These data lead us to propose that in T1D patients there is an abnormal activation of both iNKT and MAIT cells. We hypothesize that this could be due to increased levels of proinflammatory cytokines and/or increase levels of iNKT and MAIT cell ligands, which could be in turn linked to gut microbiota dysbiosis. Both cell types recognize bacterial ligands.

Hence, a specific analysis of differential markers expression on MAIT and iNKT cells from control children compared to recent onset T1D have been conducted. Those expression data for other molecules (PD1, KLRG1, Bcl2 . . . ,) have been summarized in Table 3 and some have been illustrated in FIGS. 4 to 7.

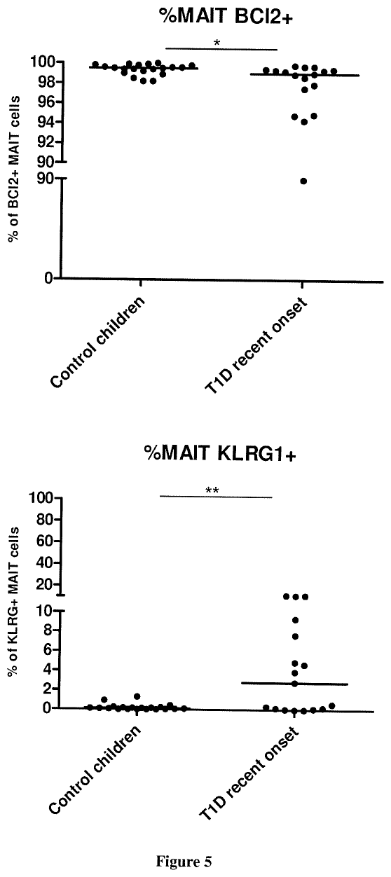

For example, as shown in FIG. 4, the quantification of PD1+, which is implicated in cell exhaustion, can be used to discriminate control children from recent T1D onset children. iNKT and MAIT cells were more often PD1-positive in patient with recent T1D, compared to controls (FIG. 4). The same can be stated for BCl2+, KLRG1+(FIG. 5).

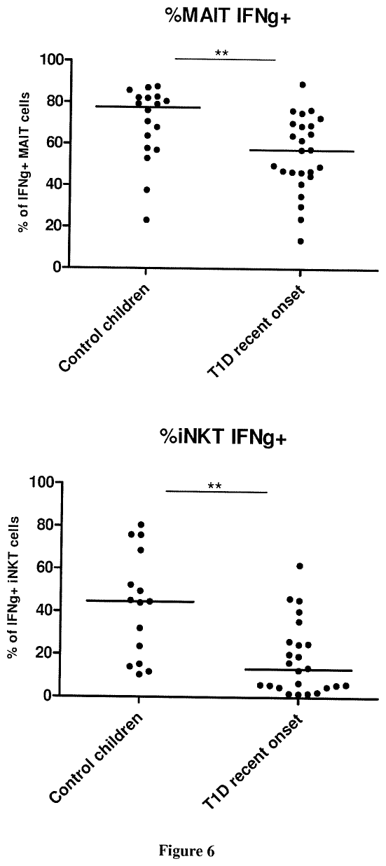

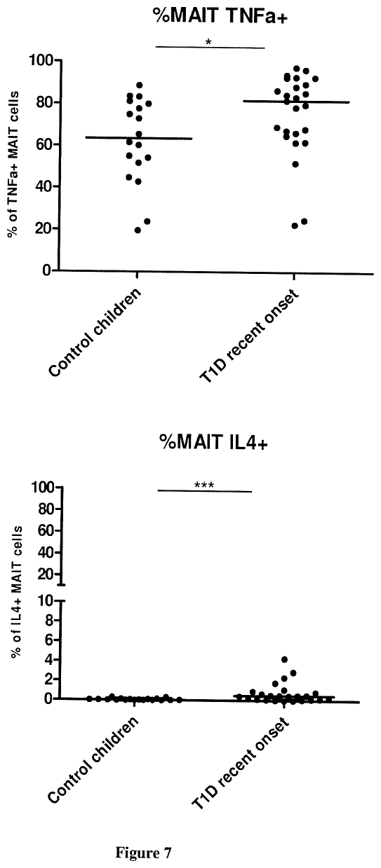

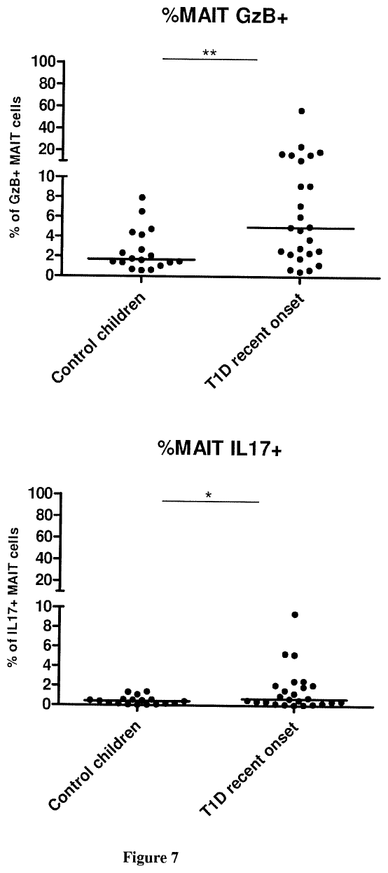

Cytokines and cytotoxic marker GzB, have also been identified, by the inventors, as relevant markers for the discrimination of recent T1D onset as shown in Table 3 and FIGS. 6 and 7.

| | | | Controls | T1D Recent |
|---|---|---|---|---|
| Test Mann-Whitney | | | children | Onset |
| EXHAUSTION | | MAIT PD1+ | − | + |
| | | MAIT KLRG1+ | −−− | + |
| | | MAIT BCL2+ | +++ | ++ |
| | | iNKT PD1+ | +/− | ++ |
| | | iNKT KLRG1+ | − | − |
| | | iNKT BCL2+ | +++ | ++ |
| CYTOTOXICITY | | MAIT GzB+ | + | +++ |
| | | iNKT GzB+ | + | + |
| CYTOKINES | Th1 | MAIT IFNg+ | +++ | ++ |
| | | MAIT TNFa+ | ++ | +++ |
| | | iNKT IFNg+ | ++ | − |
| | | iNKT TNFa+ | ++ | + |
| | Th2 | MAIT IL4+ | −−− | +++ |
| | | iNKT IL4+ | +/− | +/− |
| | Th17 | MAIT IL17+ | − | + |
| | | iNKT IL17+ | − | − |

Based on those results it can be hypothesized that the decrease of MAIT or iNKT cell frequency observed in the T1D patients and more specifically in the Children with recent onset T1D could be partially explained by their strong activation, leading in turn to their exhaustion.

We also examined the correlation between HbA1c and the proportions of CD25-positive iNKT or MAIT cells in children with recent onset T1D, but there is no correlation. It appears that in this population of patients with very early diagnosis and quite high HbA1c levels, the phenotype of interest does not seem affected by metabolic impairment. The same was observed in children with established T1D and adults with long-standing disease, which tend to have lower HbA1c levels compared to the recent onset patients. Thus, biomarkers identified by the inventors can bring more information on the phenotype of interest compared to HbA1c.

Given these data, the quantification of different population of iNKT and MAIT cells was used to discriminate control children, children with T1D recent onset and children with long standing T1D. The analysis focuses on pediatric population (81 pediatric subjects), itself composed of three sub-populations:
a population of control, composed of healthy subjects (22 subjects)
a population of newly diagnosed subjects (41 subjects) and
a population of diseased subjects (23 subjects).

With this study it was possible to refine the discrimination carried out previously on cytometric parameters and identify biological parameters which allow the best discrimination of the three sub-populations.

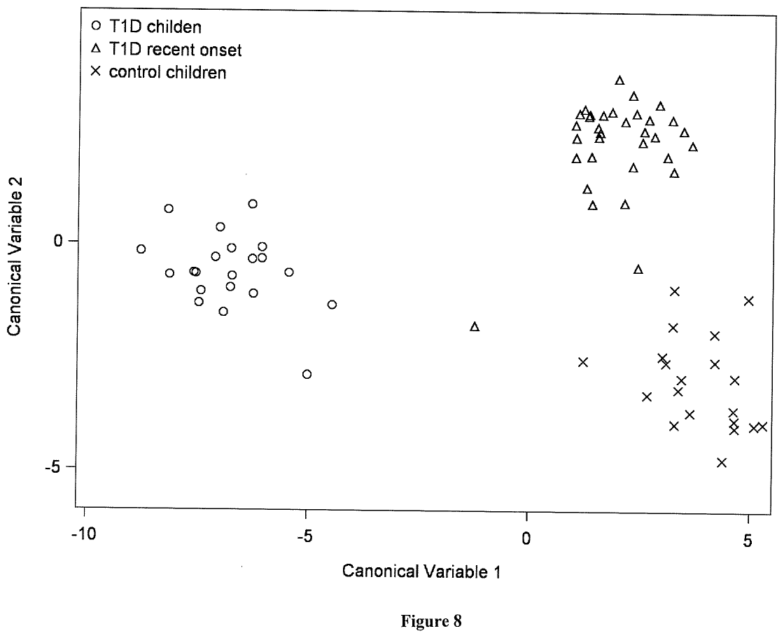

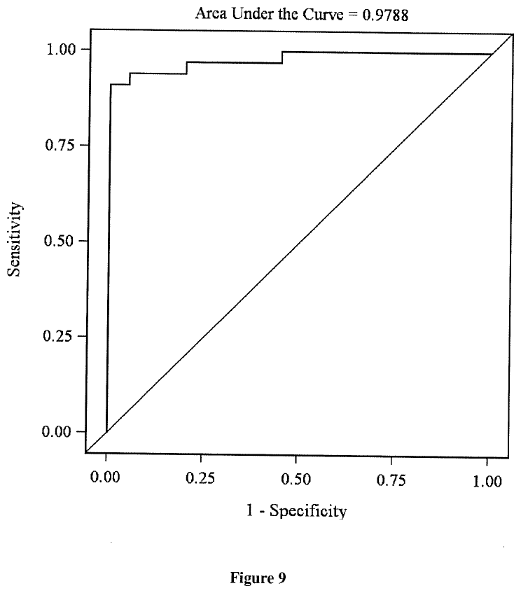

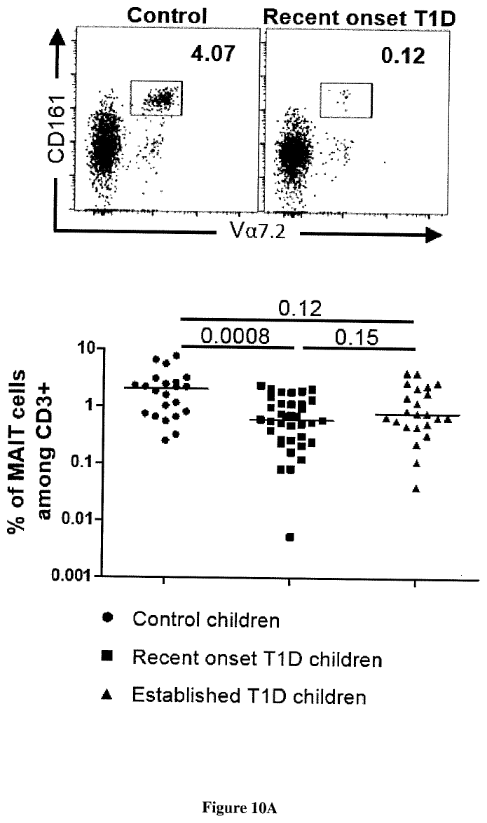

A principal component analysis (PCA) was carried out on the cytometric parameters obtained from MAIT and iNKT cells of those 81 pediatric subjects (FIG. 8). This PCA shows the 86 individuals in the factorial design (abscissa=Can1 and ordered=Can2), with their calculated coordinates. The axis Can1 allows to discriminate one hand sick children (negative values) and secondly controls children and recent diagnosis (positive values). The axis Can2 allows complete discrimination, children newly diagnosed located in the upper right quadrant (positive values, see: Table 5) and the child controls in the lower right quadrant (negative values). Long lasting T1D children are placed themselves between the two groups, close to zero.

TABLE 4
Class Means on Canonical Variables

| STATUT | Can1 | Can2 |
|---|---|---|
| T1D childen | −6.768282991 | −0.620392530 |
| T1D recent onset | 1.970238152 | 2.224445621 |
| control children | 3.855804189 | −3.018923118 |

Can1 and Can2 are based on two new variables also called component, each made as the linear combination of several cytometric parameters. The main factors of Can1 and Can2 are presented in Table 5.

TABLE 5
Raw Canonical Coefficients

| Label | Can1 | Can2 |
|---|---|---|
| MAIT CD25+ | 140.6887881 | −34.7992235 |
| MAIT CD25+ PD1+ | −836.668862 | −355.4912666 |
| MAIT CD25+ PD1− | −547.1229447 | −195.3184607 |
| MAIT CCR6+ CD25+ CD56− PD1+ | −380.5641653 | −134.9921849 |
| iNKT CCR6−CD25+CD161+ | −81.7954022 | −112.6308856 |
| MAIT CCR6+ CD25+ CD56− PD1− | −62.8455747 | −59.6811366 |
| MAIT CCR6− CD25+ PD1− | −163.9407468 | −51.5567586 |
| MAIT CCR6− CD25+ CD56− PD1− | 193.1015985 | −39.0970365 |
| MAIT CD25+ CD56− PD1+ | 495.9664932 | 72.7252498 |
| MAIT CCR6+ CD25+ CD56− | −133.1624776 | 77.4361266 |
| iNKT CCR6−CD25+CD161+PD1− | 81.8785878 | 112.9572917 |
| iNKT CCR6−CD25+CD161+PD1+ | 81.3180599 | 114.1039554 |
| MAIT CCR6+ CD25+ PD1− | 467.9625711 | 195.4004921 |
| MAIT CD25+ CCR6− | 601.7952535 | 276.8133444 |
| MAIT CCR6+ CD25+ PD1+ | 764.7454156 | 342.202342 |
| MAIT CCR6− CD25+ CD56− | −426.3194369 | 46.5641551 |
| MAIT CD25+ CD56− PD1− | 198.1796168 | −16.9507561 |

From Table 5, MAIT and iNKT populations which are highly correlated with recent T1D onset (Can2) or long lasting T1D (Can1) can be identified. It seems that cell populations defined by specific markers combination are more relevant for such discrimination.

Moreover, the analysis of the correlation of each variable with the factor axis, show that the most relevant variables on Can2 (for control vs recent onset discrimination) % MAIT (−22%), iNKT CD25+(19.1%), MAIT CCR6− PD1+(19.2%), iNKT CD25+PD1+(18.8%).

Such multifactorial analysis, based on several cytometric parameters of MAIT and iNKT cells, allow an efficient discrimination of the three pediatric populations. The representation of individuals in the factorial design (FIG. 8) is very relevant. Variable % MAIT (highest correlation with Can2 axis, and p-value=0.0003), allows to discriminate at best newly diagnosed patients and control patients; healthy individuals tend to have a higher % of MAIT individuals recently diagnosed.

Hence it seems that the study of MAIT or iNKT cells and more particularly the study of CD25, CCR6 or PD1 subpopulation is highly relevant for early diagnosis of T1D.

In order to propose a tool for the risk evaluation or early diagnosis of T1D patient, the inventors have constructed a logistic regression model based of cytometric parameters and status of patients (use of PROC LOGISTIC (SAS 9.3). A method of backward selection was used. Starting from 55 parameters used in the Discriminant Analysis, the selection method backward leads to a model that includes an intercept and variable MAIT CCR6, MAIT CD56, MAIT CCR6+CD56+, MAIT CCR6− CD56−, MAIT CD25+PD1+ and MAIT CD25+CD56− PD1+, all significant at 5% (Table 6).

TABLE 6

Wald

| Cytometric parameter | DF | Chi-SquarePr> | ChiSq |
|---|---|---|---|
| MAIT CCR6 | 1 | 5.4091 | 0.0200 |
| MAIT CD56 | 1 | 5.1571 | 0.0232 |
| MAIT CCR6+ CD56+ | 1 | 5.1552 | 0.0232 |
| MAIT CCR6− CD56− | 1 | 5.4440 | 0.0196 |
| MAIT CD25+ PD1+ | 1 | 5.8562 | 0.0155 |
| MAIT CD25+ CD56− PD1+ | 1 | 5.9922 | 0.0144 |

The ROC curve, plotted in FIG. 9, assessed the performance of the model to classify subjects based on their status (recent/healthy). The area under the curve (AUC, Area Under Curve) is 0.9788 or very close to the maximum value of 1. This curve shows the sensitivity (proportion of diagnoses classified diagnosed ordinate) versus 1−specificity (proportion of control subjects classified diagnosed).

The model confusion matrix (for a median threshold s=0.5) is presented in Table 7.4 of 53 subjects (7.5%) were misclassified: 2 and 2 control subjects newly diagnosed subjects.

TABLE 7

| | |
|---|---|
| STATUT | |
| Frequency | Predicted Statut |

| Row Pct | T1D recent onset | control children | Total |
|---|---|---|---|
| T1D recent onset | 31 | 2 | 33 |
| | 93.94 | 6.06 | |
| control children | 2 | 18 | 20 |
| | 10.00 | 90.00 | |
| Total | 33 | 20 | 53 |

Frequency Missinq = 10

Furthermore, from the model coefficients (Table 8), it is possible to diagnose a new patient, according to this data.

TABLE 8

| Variable | Coefficient | Value |
|---|---|---|
| Intercept | α | −11040.61 |
| MAIT CCR6 | β₁ | 110.368 |
| MAIT CD56 | β₂ | 105.802 |
| MAIT CCR6+ CD56+ | β₃ | −105.708 |
| MAIT CCR6− CD56− | β₄ | 111.147 |
| MAIT CD25+ PD1+ | β₅ | −96.8976 |
| MAIT CD25+ CD56− PD1+ | β₆ | 154.676 |

Such diagnosis can be made using the following logistic regression based formula, with 71 the probability of a subject to be diagnosed sick:

$$logit ( π ) = log ( \frac{π}{1 - π} ) = α + {β}_{1} \times MAIT CCR 6 + {β}_{2} \times MAIT CD 56 + {β}_{3} \times MAIT CCR {6}_{+} CD {56}_{+} + {β}_{4} \times MAIT CCR {6}_{-} CD {56}_{-} + {β}_{5} \times MAIT CD {25}_{+} PD {1}_{+} + {β}_{6} \times MAIT CD {25}_{+} CD {56}_{-} PD {1}_{+}$$

Noting X′β the previous result

$$π = \frac{exp ( {X}^{′} β )}{1 + exp ( {X}^{′} β )}$$

For example, two hypothetical patients were defined (Table 9). Given their respective values for the values of the model, the patient would be diagnosed patient A (π≥0.6), and the patient would be diagnosed healthy B (π<0.6).

TABLE 9

| Sujet | Variable | Valeur | X′β | π |
|---|---|---|---|---|
| Pa- | MAIT CCR6 | 95 | 1.042 | 0.73932 |
| tient | MAIT CD56 | 10 | | |
| A | MAIT CCR6+ CD56+ | 10 | | |
| | MAIT CCR6− CD56− | 5 | | |
| | MAIT CD25+ PD1+ | 0 | | |
| | MAIT CD25+ CD56− PD1+ | 0 | | |
| Pa- | MAIT CCR6 | 95 | −331.459 | <0.0001 |
| tient | MAIT CD56 | 20 | | |
| B | MAIT CCR6+ CD56+ | 20 | | |
| | MAIT CCR6− CD56− | 2 | | |
| | MAIT CD25+ PD1+ | 0 | | |
| | MAIT CD25+ CD56− PD1+ | 0 | | |

Such model have also been used on a data set which has not been used for the regression model construction (Table 10).

TABLE 10

| Statut | Variables | Values | X′β | π |
|---|---|---|---|---|
| T1D recent onset | MAIT CCR6+ | 97.7 | 8.8087808 | 0.99985061 |
| | MAIT CD56+ | 17.7 | | |
| | MAIT CCR6+ CD56+ | 17.5 | | |
| | MAIT CCR6− CD56− | 2.16 | | |
| | MAIT CD25+ PD1+ | 0.062 | | |
| | MAIT CD25+ CD56− PD1+ | 0.062 | | |
| T1D recent onset | MAIT CCR6+ | 94.5 | 367.497568 | 1 |
| | MAIT CD56+ | 11 | | |
| | MAIT CCR6+ CD56+ | 11 | | |
| | MAIT CCR6− CD56− | 5.48 | | |
| | MAIT CD25+ PD1+ | 8.22 | | |
| | MAIT CD25+ CD56− PD1+ | 7.53 | | |
| control | MAIT CCR6+ | 98.6 | −13.94301 | 8.8029E−07 |
| | MAIT CD56+ | 40.1 | | |
| | MAIT CCR6+ CD56+ | 40 | | |
| | MAIT CCR6− CD56− | 1.17 | | |
| | MAIT CD25+ PD1+ | 0 | | |
| | MAIT CD25+ CD56− PD1+ | 0 | | |
| control | MAIT CCR6+ | 92.9 | −2.9174316 | 0.05129855 |
| | MAIT CD56+ | 33 | | |
| | MAIT CCR6+ CD56+ | 32.2 | | |
| | MAIT CCR6− CD56− | 6.23 | | |
| | MAIT CD25+ PD1+ | 0.076 | | |
| | MAIT CD25+ CD56− PD1+ | 0.076 | | |

Such results confirmed the effectiveness of the quantification of MAIT or iNKT cells and more particularly their subpopulation for the follow up of T1D pathology and more particularly it early diagnosis.

To conclude, dysregulation of iNKT and MAIT cells may occur time before diabetes diagnosis, during the prediabetes period and might modify the balance between the activating and inhibiting ligands of MAIT and iNKT cells in such a way that both cell types could be activated before the multiple seroconversion state.

### EXAMPLE 2

Material and Methods

Human samples. Peripheral blood samples were obtained from control children and from T1D children admitted in the Pediatric Endocrinology department of Necker hospital, Paris, France at T1D onset (i.e. within 10 days from first insulin injection), or with established disease. None of the control children had a personal or familial history of T1D or autoantibodies associated with T1D. Non-inclusion clinical following parameters contain: infection during the admission and associated others autoimmune disease. The Ethics Committee (Comité de protection des personnes (CPP) Ile-de-France) approved the clinical investigations and written informed consent was obtained from all the parents. For the Milan cohort, peripheral blood from healthy control subjects and patients at T1D onset (i.e., within 10 days from first insulin injection) were collected. The study was approved by the San Raffaele Hospital Ethic Committee (protocol: DRI-003). At risk subjects were enrolled in the Type 1 Diabetes TrialNet Pathway to Prevention Trial (TNO1 trial, former TrialNet Natural History Study). The overall objective of this study is to perform baseline and repeated assessments over time of the immunologic and metabolic status of individuals who are at risk for T1D (first/second degree relatives of patients with T1D). The study was approved by the San Raffaele Hospital Ethics Committee (protocol: NHPROTOCOL32803 TN01). Our local study was approved by the TrialNet Ancillary Studies Subcommittee. All analyses were performed blinded and all subjects included in this study signed the informed consent prior to blood donation.

Cell preparations. Human peripheral blood mono-nucleated cells (PBMC) of patients from Necker Hospital were isolated from fresh blood samples by Ficoll-Paque (Leucosep) or samples from San Raffaele hospital were defrosted in RPMI with 10% FCS (Fetal Cow Serum).

Flow cytometry and antibodies. Cells were stained in PBS containing 5% FCS and 0.1% azide. For human PBMC the following antibodies were used: CD3 (OKT3), CD4 (OKT4), Vα7.2 (3C10), CD161 (HP-3G10), CCR6 (G034E3), CD56 (HCD56), CD69 (FN50), Bcl-2 (100), IFN-γ (4S B3), IL17A (BL168), TNF-α (MAb11) mAbs from BioLegend; CD8 (SK1), PD1 (MIH4), CD25 (M-A251), IL4 (8D4-8), granzyme B (GB11) mAbs from BD Biosciences; CD3 (REA), CD161 (REA) mAbs from Miltenyi and CD4 (RPA-T4) mAb from eBiosciences. Data acquisition was performed using BD Biosciences LSRFortessa cytometer or FACS ARIA III cytometer for cells from patients from Necker hospital and Beckman Coulter Gallios for cells from patients from San Raffaele hospital.

In vitro cell stimulation. For ligand stimulation of human MAIT cells, 5-OP-RU solution was obtained after incubating 1 molar equivalent of 5-ARU with 2 molar equivalent of methylglyoxal (Sigma-Aldrich). Hela cells and PBMC from children controls and children with recent onset T1D were plated at a final concentration of 500 000 cells/mL each on 24-well plate, in RPMI supplemented with 10% FCS from children controls and children with recent onset T1D were plated at a final concentration of 106 cells/mL in RPMI supplemented with 10% FCS. PBMC were stimulated ON with various concentration of 5-OP-RU (0-5 nmol/L). Blocking MR1 (26.5 mAb) from Biolegend was added when indicated at 10 μg/mL. MAIT cell activation was analyzed by flow cytometry.

Intra-cellular staining. For human Bcl-2 staining, after surface staining lymphocytes were resuspended in fixation/permeabilization buffer (Foxp3 staining kit from eBioscience) and incubated at 4° C. in the dark then, cells were washed with PERM Wash buffer (eBioscience) and labeled with appropriate mAbs. For cytokine and granzyme B analysis of human MAIT cells, PBMC obtained from fresh samples, were analyzed after stimulation for 6 h at 37° C. in RPMI medium supplemented with 10% FCS with PMA (25 ng/ml) and ionomycin (1 μg/ml), in the presence of Brefeldin A (10 μg/ml).

Statistical analysis. For human studies, statistical tests between two groups were performed using two-tailed Mann-Whitney test and signed-rank Wilcoxon test with Graph Pad Prism. The Kruskal-Wallis test followed by the Wilcoxon rank sum test adjusted with the Holm method and the Spearman correlation test was applied for all the correlation analysis with Software R. A factorial discriminant analysis was performed using the XLSTAT 2016 Software. A logistic regression model was fitted with the PROC CANDISC software (SAS version p. 3) then a backward elimination procedure was applied. Prognostic validity of the model was evaluated by the receiver operating characteristic (ROC) curve analysis and measured using the area under the ROC curve (AUC). Statistical analyses were performed using the GraphPad Prism software version 5.00.288 and the R software version 3.2.3.

Results

Alteration of Blood MAIT Cell Frequency and Phenotype in Children with Recent Onset T1D

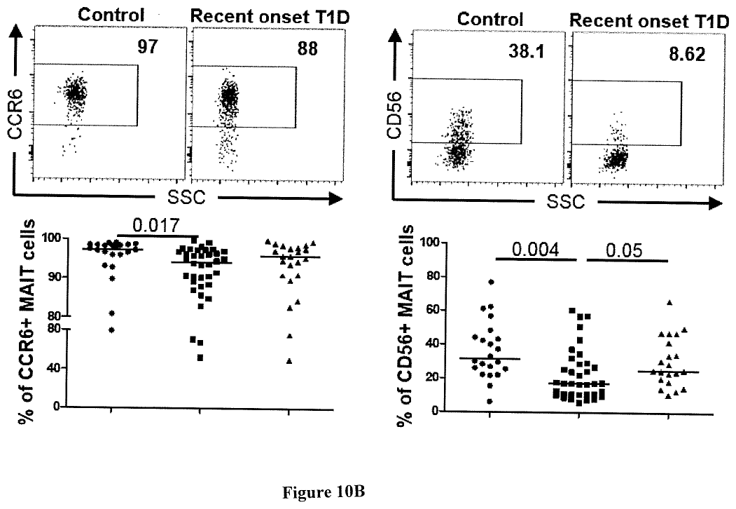

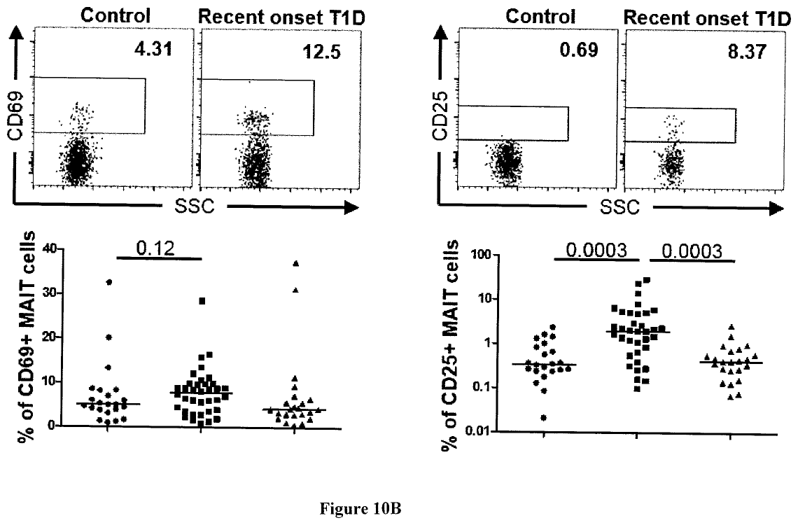

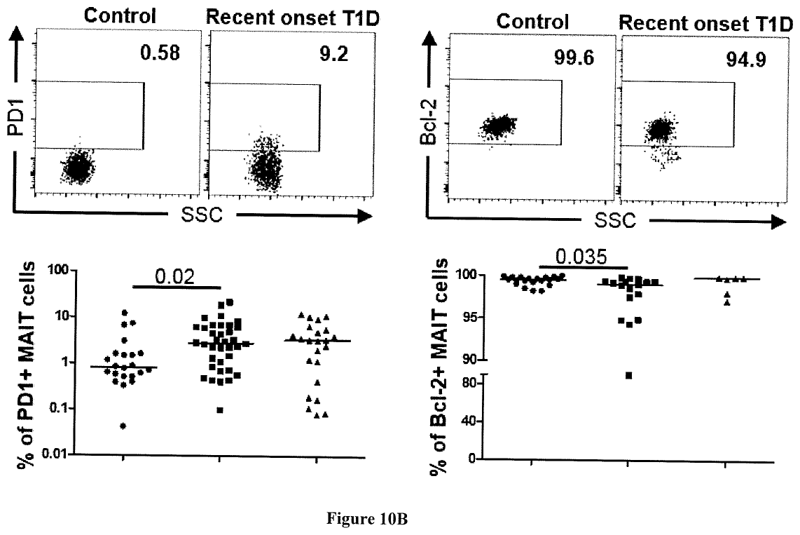

We first began the investigation of MAIT cells in T1D by analyzing MAIT cell frequency and phenotype in fresh peripheral blood samples from children with recent onset T1D and children with established T1D as compared to age-matched control children (data not shown). MAIT cells can be identified in human blood as CD4⁻ T lymphocyte expressing Vα7.2 TCRα gene segment and CD161^{high} (FIG. 10a). MAIT cell frequency and number was decreased (3-fold) in the blood of recent onset T1D children whereas no significant difference was observed in children with established disease as compared to control children (FIG. 10a and data not shown). Decreased frequency was observed in both CD8+ and double negative (DN) MAIT cell subsets (data not shown). Of note there was no difference in the frequencies of conventional CD4 and CD8 T cells, and of Vα7.2⁺CD161⁻ T cells between the three children populations confirming that the decrease of MAIT cell frequency at the onset of T1D was not consecutive of changes in other T cell populations nor to down-regulation of the CD161 marker (data not shown). Analysis of MAIT cell phenotype showed a decreased frequency of MAIT cells expressing tissue recruitment/adhesion molecules (CCR6, CD56) at the onset of the disease, an increased frequency of MAIT cells expressing the activation/exhaustion markers CD25 and PD1, and a decreased frequency of MAIT cells expressing the anti-apoptotic molecule Bcl-2 (FIG. 10b and data not shown). Multi-parametric analysis of MAIT cells in the children with established T1D highlighted the intermediate phenotype of MAIT cells between those from recent onset T1D and control children (data not shown). Interestingly in recent onset children the frequency of MAIT cells expressing migratory CCR6⁺ or anti-apoptotic Bcl-2 molecules were positively correlated with the frequency of MAIT cells, whereas MAIT cell CD25 expression was negatively correlated with MAIT cell frequency (data not shown). These data suggest that decreased blood MAIT cell frequency could reflect their migration to inflamed tissues and/or their death by apoptosis subsequent to their activation.

Alteration of Blood MAIT Cell Function in Children with Recent Onset T1D

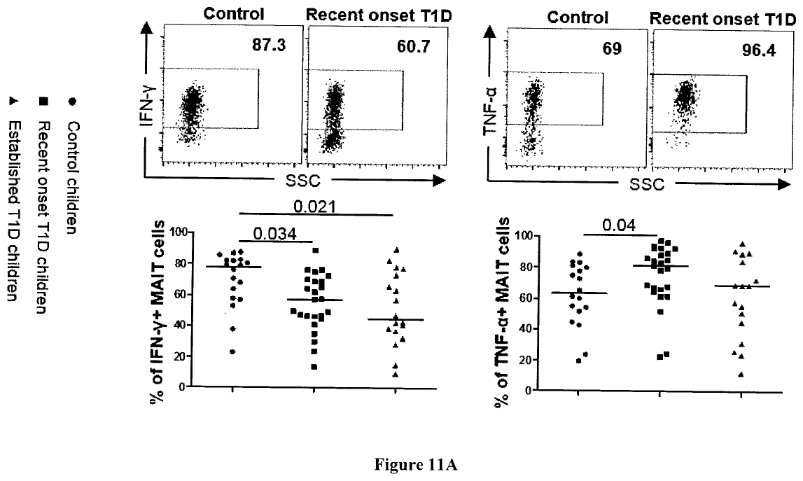

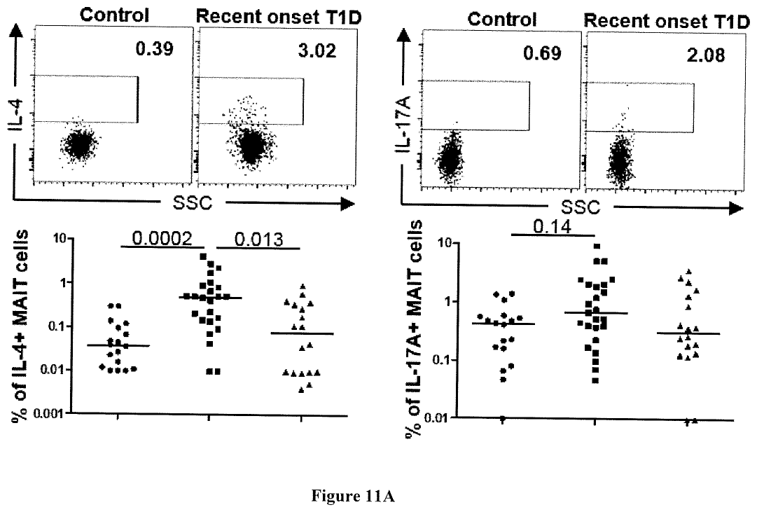

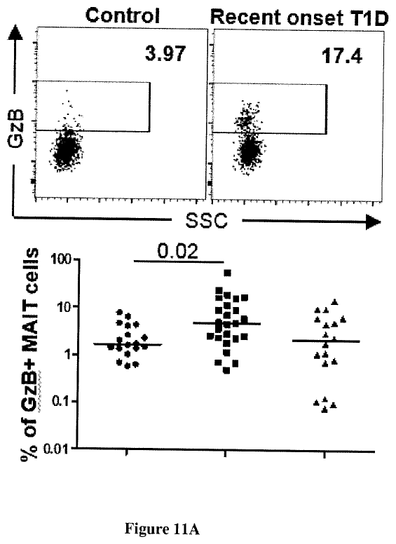

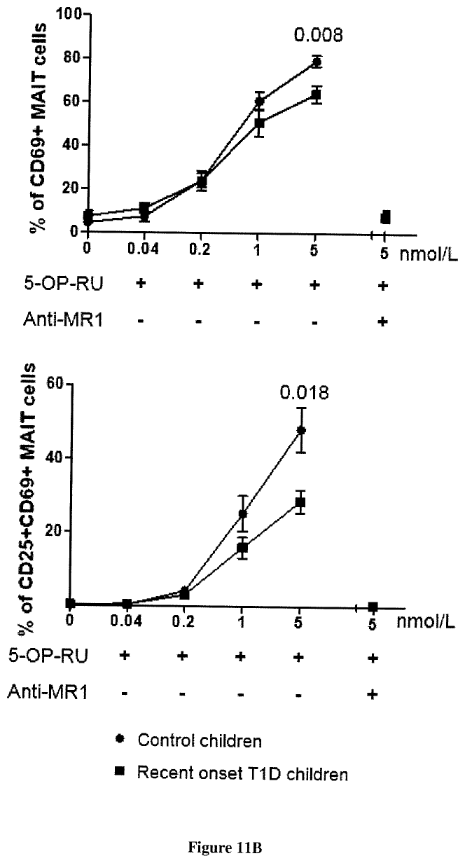

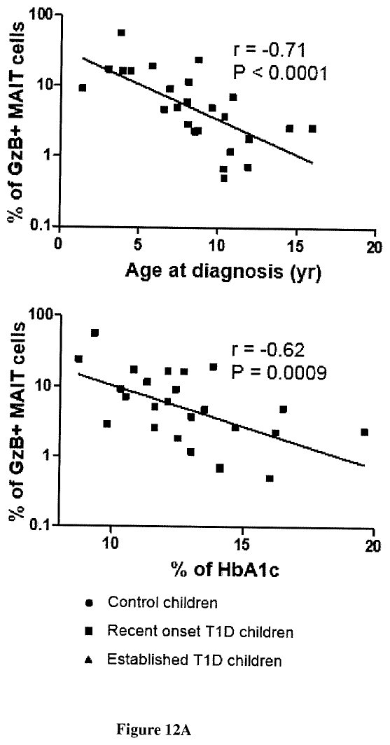

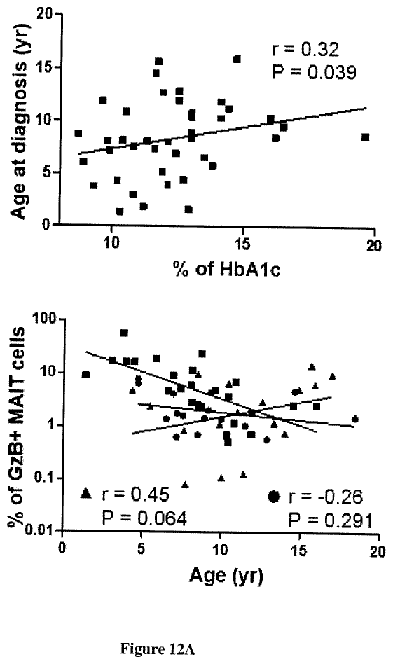

Cytokine and GzB production by fresh blood MAIT cells was analyzed after PMA-ionomycin stimulation. MAIT cells from children with recent onset T1D produced less IFN-γ, whereas their production of TNF-α, IL-4, and GzB was increased as compared with MAIT cells from control children (FIG. 11a and data not shown). Of note, among these effector molecules only the frequency of GzB correlated with the frequency of MAIT cells, the higher GzB production was observed in patients with the lower frequency of MAIT cells (data not shown). Multi-parametric analysis of cytokines and GzB production by MAIT cells also showed an intermediate status of blood MAIT cells from children with established T1D, between those from control children and recent onset T1D children, as already observed for MAIT cell surface phenotype (data not shown). We next analyzed the ability of MAIT cells to respond to specific TCR activation by the ligand 5-OP-RU. Upon stimulation, MAIT cells from control children up-regulated CD69 and CD25 activation markers. Addition of blocking MR1 mAb confirms that this activation was TCR-dependent. Interestingly, MAIT cell activation was significantly reduced in children with recent onset T1D (FIG. 11b). Together, these results highlight functional alteration of MAIT cells in children with recent onset T1D.

Association Between MAIT Cell Alterations and Clinical Characteristics

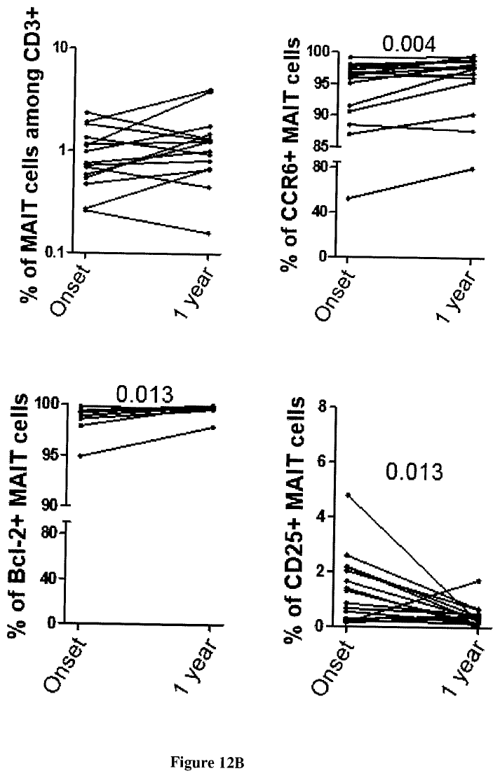

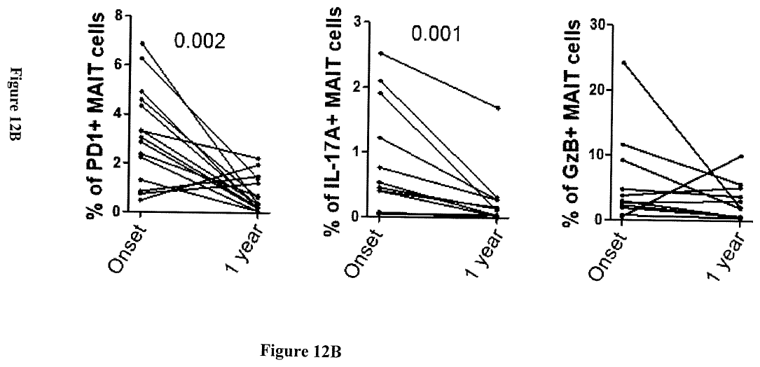

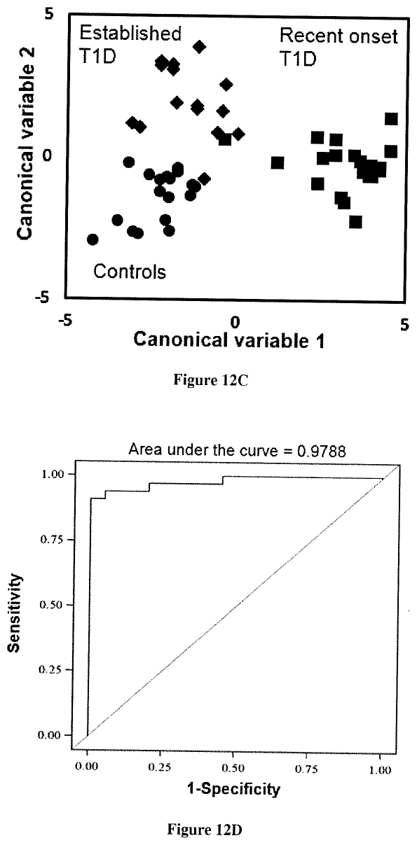

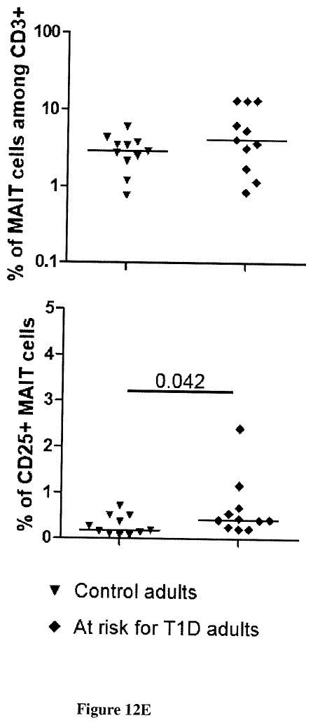

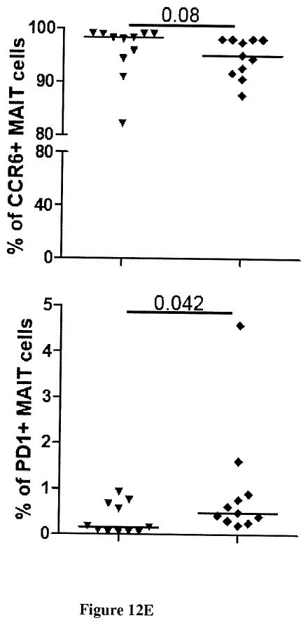

We next investigated potential links between phenotype and functional alterations of MAIT cells and clinical characteristics of children with recent onset T1D (data not shown). Interestingly, the frequency of GzB⁺ MAIT cells was negatively associated (r=−0.71, P<0.0001) with children's age at diagnosis (FIG. 12a), which is in agreement with the current view that T1D is more aggressive in the youngest children. We speculate that production of GzB by MAIT cells, reflecting their cytotoxic potential, could be involved in the physiopathology of T1D. Other MAIT cell parameters, such as their frequency, CCR6 and Bcl-2 expression, were also associated with the age at diagnosis (data not shown). No significant correlations between MAIT cell parameters and age were observed in controls and children with established T1D (data not shown). Production of GzB by MAIT cells inversely correlated with HbA1c level at the onset of the disease but not in children with established T1D (FIG. 12a). Indeed, a more aggressive disease associated with sustained MAIT cell abnormalities suggests a shorter time of hyperglycemia before the onset thereby lower levels of HbA1c.

To further explore the link between MAIT cell parameters and clinical characteristics, 15 of the children with recent onset T1D were further analyzed one year later. Although MAIT cell frequency among T cells remain to a similar level after one year of insulin, both CCR6+ and Bcl-2⁺ MAIT cell frequencies significantly increased. Conversely the frequencies of CD25+, PD1+, IL-17A⁺, and to some extent GzB⁺, MAIT cells decreased to levels observed in the control children (FIG. 12b). This longitudinal analysis strengthened the data obtained in the transversal analysis showing that MAIT cell phenotype was more similar to controls in the children under insulin therapy than at disease onset.

MAIT Cells as a New Biomarker of T1D

Canonical analysis was performed to compare MAIT cell alterations in the three groups of children (controls, recent onset and established T1D). This analysis revealed that MAIT cell parameters were sufficient to discriminate the three groups of children analyzed (FIG. 12c). Moreover a statistical regression analysis identified four surface markers that define a predictive model for the diagnosis of the disease tested on the ROC curve (FIG. 12d).

Importantly, we confirmed in another cohort of children from Milano that frequency and phenotype alteration of MAIT cell was observed in recent onset T1D as compared to age-matched control children (data not shown). However technical difficulties impacting CD56 analysis on these frozen cells did not allow to applying the predictive model.

Finally, to test whether MAIT cell alterations could be detected before the onset of diagnosis, we characterized MAIT cells in adults at risk to develop T1D defined as direct relatives of T1D patients with at least two positive autoantibodies (data not shown). As compared to the control group, there were increased frequencies of CD25⁺ and PD1⁺ MAIT cells and a trend toward a lower CCR6⁺ MAIT cell frequency, even though the number of individuals analyzed were limited (FIG. 12e). Altogether our data in patients suggest that MAIT cells represent a new biomarker in T1D and they could play a role in the pathogenesis of T1D. Therefore we investigated MAIT cells in mouse models, which allow analysis in tissues at different stages of disease development and could be manipulated to determine whether MAIT cells are involved in T1D physiopathology.

### REFERENCES

Throughout this application, various references describe the state of the art to which this invention pertains. The disclosures of these references are hereby incorporated by reference into the present disclosure.

1. A method of treating a child who has or is at risk of having type 1 diabetes (T1D), comprising:
measuring in a blood sample obtained from a child, a quantity of at least 2, 3, 4, 5, 6, 7, 8, 9, or 10 populations of Mucosal-Associated Invariant T (MAIT) cells selected from the group consisting of: MAIT CCR6+ cells, MAIT CD25+ cells, MAIT CD69+ cells; MAIT CD56+ cells, MAIT PD1+ cells, MAIT TIM3+ cells, MAIT KLRG1+ cells, MAIT BTLA+ cells, MAIT BCL2+ cells, MAIT Ki67+ cells, MAIT GzB+ cells, MAIT IFNg+ cells, MAIT TNFa+ cells, MAIT IL4+ cells, MAIT IL10+ cells, MAIT KLRG1+ cells and MAIT IL17+ cells, wherein measurements made on the blood sample of the child indicate at least one of
a quantity of MAIT CCR6+ cells in the blood sample is lower a predetermined reference value determined for control children that do not have a personal or familial history of T1D or autoantibodies associated with T1D,
a quantity of MAIT CD25+ cells in the blood sample is higher than a predetermined reference value for control children that do not have a personal or familial history of T1D or autoantibodies associated with T1D,
a quantity of MAIT CD69+ cells in the blood sample is higher than a predetermined reference value for control children that do not have a personal or familial history of T1D or autoantibodies associated with T1D,
a quantity of MAIT CD56+ cells in the blood sample is lower than a predetermined reference value for control children that do not have a personal or familial history of T1D or autoantibodies associated with T1D,
a quantity of MAIT PD1+ cells in the blood sample is higher than a predetermined reference value for control children that do not have a personal or familial history of T1D or autoantibodies associated with T1D
a quantity of MAIT TIM3+ cells in the blood sample is higher than a predetermined reference value for control children that do not have a personal or familial history of T1D or autoantibodies associated with T1D,
a quantity of MAIT KLRG1+ cells in the blood sample is higher than a predetermined reference value for control children that do not have a personal or familial history of T1D or autoantibodies associated with T1D,
a quantity of MAIT BTLA+ cells in the blood sample is higher than a predetermined reference value for control children that do not have a personal or familial history of T1D or autoantibodies associated with T1D,
a quantity of MAIT BCL2+ cells in the blood sample is lower than a predetermined reference value for control children that do not have a personal or familial history of T1D or autoantibodies associated with T1D,
a quantity of MAIT Ki67+ cells in the blood sample is higher than a predetermined reference value for control children that do not have a personal or familial history of T1D or autoantibodies associated with T1D,
a quantity of MAIT GzB+ cells in the blood sample is higher than a predetermined reference value for control children that do not have a personal or familial history of T1D or autoantibodies associated with T1D,
a quantity of MAIT IFNg+ cells in the blood sample is lower than a predetermined reference value for control children that do not have a personal or familial history of T1D or autoantibodies associated with T1D,
a quantity of MAIT TNFa+ cells in the blood sample is higher than a predetermined reference value for control children that do not have a personal or familial history of T1D or autoantibodies associated with T1D,
a quantity of MAIT IL4+ cells in the blood sample is higher than a predetermined reference value for control children that do not have a personal or familial history of T1D or autoantibodies associated with T1D,
a quantity of MAIT IL10+ cells in the blood sample is higher than a predetermined reference value for control children that do not have a personal or familial history of T1D or autoantibodies associated with T1D,
a quantity of KLRG1+ cells in the blood sample is higher than a predetermined reference value for control children that do not have a personal or familial history of T1D or autoantibodies associated with T1D, and
a quantity of MAIT IL17+ cells in the blood sample is higher than a predetermined reference value for control children that do not have a personal or familial history of T1D or autoantibodies associated with T1D; and
administering insulin to the child after measurements are made on the blood sample of the child in the measuring step.

2. The method of claim 1 further comprising measuring in the blood sample of the child at least one population of iNKT cells characterized by the presence or absence of at least one marker selected from the group consisting of CCR6, CD56, CD25, CD69, CD161, CD27, PD1, TIM3, KLRG1, BTLA, BCL2, Ki67, CD127, GzB, IFNg, IL2, TNFa, IL4, IL10 and IL17.

3. The method of claim 2 wherein the at least one population of iNKT cells is selected from the group consisting of iNKT BTLA+ cells, iNKT CCR6+ cells, iNKT CD25+ cells, iNKT CD56+ cells, iNKT CD69+ cells, iNKT CD127+ cells, iNKT CD161+ cells, iNKT PD1+ cells, iNKT CD27− cells, iNKT KLRG1+, and iNKT TIM3+ cells.

4. The method of claim 1, wherein the child is 3 to 5 years old.

5. The method of claim 1 further comprising administering to the child one or more agents that control blood glucose levels and/or increase insulin production.

6. A method treating an adult who has or is at risk of having type 1 diabetes (T1D), comprising:
measuring in a blood sample obtained from the adult, a quantity of at least 2, 3, 4, 5, 6, 7, 8, 9, or 10 populations of MAIT cells selected from the group consisting of: MAIT CD25+ cells, MAIT CD69+ cells, MAIT CD56+ cells, MAIT Ki67+ cells, MAIT IFNg+ cells and MAIT TNFa+ cells, wherein measurements made on the blood sample of the adult indicate at least one of
a quantity of MAIT CD25+ cells in the blood sample is higher than a predetermined reference value for control adults that do not have TID,
a quantity of MAIT CD69+ cells in the blood sample is higher than a predetermined reference value for control adults that do not have TID,
a quantity of MAIT CD56+ cells in the blood sample is higher than a predetermined reference value for control adults that do not have TID,
a quantity of MAIT Ki67+ cells in the blood sample is higher than a predetermined reference value for control adults that do not have TID,
a quantity of MAIT IFNg+ cells in the blood sample is lower than a predetermined reference value for control adults that do not have TID, and
a quantity of MAIT TNFa+ cells in the blood sample is lower than a predetermined reference value for control adults that do not have TID; and
administering insulin to the adult after measurements are made on the blood sample of the adult in the measuring step.

7. The method of claim 6 further comprising measuring in the blood sample of the adult a quantity of iNKT cells characterized by the presence or absence of at least one marker selected from the group consisting of CCR6, CD56, CD25, CD69, CD161, CD27, PD1, TIM3, KLRG1, BTLA, BCL2, Ki67, CD127, GzB, IFNg, IL2, TNFa, IL4, IL10 and IL17.

8. The method of claim 6 wherein the adult is more than 18 years old.

9. The method of claim 6 further comprising administering to the adult one or more agents that control blood glucose levels and/or increase insulin production.
